(19) United States
(12) Patent Application Publication
(10) Pub. No.: US 2011/0059106 A1
(43) Pub. Date: Mar. 10, 2011

(54) METHODS FOR MODULATING A POPULATION OF MYELOID-DERIVED SUPPRESSOR CELLS AND USES THEREOF

(75) Inventors: Vijay K. Kuchroo, Newton, MA (US); Ana Carrizosa Anderson, Brookline, MA (US); Valerie Dardalhon, Montpellier (FR)

(73) Assignee: BRIGHAM AND WOMEN'S HOSPITAL, INC., Boston, MA (US)

(21) Appl. No.: 12/865,322

(22) PCT Filed: Jan. 29, 2009
(86) PCT No.: PCT/US09/32372

§ 371 Date: Nov. 23, 2010

Related U.S. Application Data
(60) Provisional application No. 61/024,228, filed on Jan. 29, 2008.

(51) Int. Cl. A61K 39/395; A61K 38/17; A61P 37/06; A61P 35/00

(52) U.S. Cl. 424/158.1; 514/20.9

(57) ABSTRACT

The invention provides for methods of modulating a population of myeloid-derived suppressor cells (MDSCs) in a subject, for treating an autoimmune disease, and also treating cancer in a subject in need thereof. The methods comprise administering to the subject a therapeutically effective amount of an agent that modulates the Tim-3 pathway. The Tim-3 pathway can be activated by a Tim-3 ligand, galectin-9, whereby MDSCs are expanded, or inhibited by an antibody to Tim-3, wherein the expansion of MDSCs is inhibited.

### CROSS REFERENCE TO RELATED APPLICATION

This application claims benefit under 35 U.S.C. §119(e) of the U.S. provisional application No. 61/024,228 filed Jan. 29, 2008, the contents of which are incorporated herein by reference in its entirety.

### GOVERNMENT SUPPORT

This invention was made with Government support under Grant No.: NS38037 and NS045937 awarded by the National Institutes of Health. The Government has certain rights in the invention.

### BACKGROUND OF INVENTION

Myeloid suppressor cells, also known as myeloid-derived suppressor cells (MDSCs), accumulate in large numbers in cancer patients and they have potent immunosuppressive functions. Activation and expansion of MDSCs have been shown to be initiated in response to the cytokine, IFN-γ, that is produced by the anti-tumor T-cells in the tumor microenvironment. The failures of many cancer immunotherapies have been attributed to the accumulation of large numbers MDSCs during the immunotherapy. Hence, there is a need for methods of blocking the activation and expansion of MDSCs during such therapies.

In mice, the MDSCs express the macrophage/monocyte marker CD11b and the granulocyte marker Gr-1 (Ly-6G) but lack expression of mature antigen presenting cell (APC) markers including MHC Class II and F480. The expansion of MDSCs has also been observed after exposure to bacterial, parasitic, and viral antigens and after traumatic stress. In view of their immature cell surface phenotype and their ability to mediate immune suppression, these cells have been termed myeloid-derived suppressor cells (MDSCs).

Several mechanisms of immune suppression have been ascribed to MDSCs, including increased Arginase metabolism by Arginase1 and the production of reactive oxygen species and peroxynitrites by inducible nitric oxide synthase (iNOS)7. IFN-γ, is intimately associated with MDSCs in that they fail to accumulate in IFN-γ R^{-/-} mice and require IFN-γ for activating suppressor functions.

On the other hand, suppression of the immune system can be desirable in autoimmune diseases and in organ transplant situations, wherein an overactive immune response can cause great permanent damage to the afflicted individual and or donor organ. In view of the biological importance of MDSCs in regulating immune responses, finding novel strategies that modulate their expansion and/or activation can provide for the prevention and/or treatment of cancer, autoimmune diseases, host-graft-rejection and also pathogen infections.

### SUMMARY OF THE INVENTION

The invention provides for methods of modulating a population of myeloid-derived suppressor cells (MDSCs) in a subject. In one embodiment, the method comprises administering to the subject a therapeutically effective amount of an agent that modulates the Tim-3 pathway.

As used herein, modulation of a population of MDSC comprises promoting the expansion of MDSCs or inhibiting the expansion of MDSCs.

As used herein, modulation of the Tim-3 pathway comprises activating the Tim-3 pathway wherein there is an increase of tyrosine phosphorylation in the cytoplasmic portion of the Tim-3 protein and this leads to further downstream signaling events characteristic of the pathway, which includes promoting an expansion of MDSCs. Activating the Tim-3 pathway comprises binding of the Tim-3 receptor protein with a specific ligand, e.g. galectin-9. Modulation of the Tim-3 pathway can also comprise inhibiting the Tim-3 pathway wherein there is an decrease or no tyrosine phosphorylation in the cytoplasmic portion of the Tim-3 protein and reduced or no downstream signaling events characteristic of the pathway, which includes inhibiting an expansion of MDSCs. Inhibiting the Tim-3 pathway comprises blocking binding of the Tim-3 receptor protein with a ligand, e.g. blocking with an anti-Tim-3 antibody.

In one embodiment, the modulation of the population of MDSCs can take the form of promoting the expansion of MDSCs. In one embodiment, the agent that modulates the Tim-3 pathway activates the Tim-3 pathway. Activating the pathway results in phosphorylation of Tim-3 and/or downstream signaling cascade events. Since MDSCs are potent immunosuppression cells, expansion of MDSCs leads to suppression of the immune system, in particular, the adaptive immune response.

Agents that can activate the Tim-3 pathway include, for example, galectin-9, galectin-9 analog, fragments, and derivatives, a galectin-9 peptido-mimetic, an activating Tim-3 antibody, or a vector expressing Tim-3 or galectin-9.

The suppression of the immune system, in particular, the adaptive immune response, is highly desirable in subjects, for example, suffering from an autoimmune disease or having undergone an organ transplant. In all automimmune diseases, a dysfunctional or overactive adaptive immune system is the underlying cause of the disease; therefore, suppression of the immune system can treat the disease and ameliorate the symptoms. Suppression of the immune system is also desirable for the long-term viability of the donor organ in the host.

In another embodiment, the modulation of the population of MDSCs can also take the form of preventing the expansion of MDSCs. In one embodiment, the agent that modulates the Tim-3 pathway inhibits the Tim-3 pathway. Inhibiting the pathway blocks downstream signaling cascade events necessary for the expansion of MDSCs. Fewer MDSCs relieves the suppression of the immune system.

Agents that inhibit the Tim-3 pathway include, for example, antibodies against Tim-3, specifically reactive to the extracellular region of Tim-3, such as the antibodies from the hydridomas 8B.2C12 and 25F.1D6. Alternately, the agent can be soluble Tim-3 proteins or conjugates, galectin-9-binding molecule or protein or antibody, small interfering RNA specific for or target to Tim-3 or galectin-9, and antisense RNA that hybridizes with the messenger RNA of Tim-3 or galectin-9.

Relieving the suppression of the immune system is highly desirable for an immunocompromised subject or a subject suffering from cancer. In the presence of fewer MDSCs, the immune system can be properly simulated to attack cancer cells and foreign infectious pathogens such as bacteria and viruses.

The invention also provides for a method of treating an autoimmune disease in a subject. In one embodiment, the method comprises expanding a population of MDSCs in a subject by activating the Tim-3 pathway. In one embodiment, the Tim-3 pathway is activated by galectin-9.

The invention also provides for a method of treating cancer in a subject. In one embodiment, the method comprises inhibiting the expansion of a population of MDSCs in a subject by inhibiting the Tim-3 pathway. In one embodiment, the Tim-3 pathway is inhibited by a Tim-3 antibody such as antibodies from hydridomas 8B.2C12 and 25F.1D6.

In one embodiment, the MDSCs are characterized by the macrophage/monocyte marker CD11b+ and granulocyte marker Gr-1/Ly-6G high, and having low or undetectable expression of the mature antigen presenting cells markers MHC Class II and F480.

### BRIEF DESCRIPTION OF THE DRAWINGS

FIG. 1 shows the Tim-3 expression in Tim-3-Tg mice (open histogram) relative to wild type littermate controls (shaded histogram) in CD4 single positive and CD8 single positive (CD4SP and CD8SP) thymocytes and peripheral CD4+ and CD8+ T cells.

FIG. 2A shows the CD44 expression on CD4+ spleen cells from naïve wild type and Tim-3-Tg mice.

FIG. 2B shows the CD44 hi/lo ratio in Tim-3-Tg (n=7) versus wild type (n=7) littermates, (p=0.0023, Mann-Whitney U test).

FIG. 3A shows that splenocytes (5×10⁵/well) from naïve wild type and Tg mice stimulated with soluble anti-CD3. Proliferated is measured by [³H]-thymidine incorporation. Error bars indicate s.e.m.

FIG. 3B shows the IFN-γ production from naïve wild type and Tg mice stimulated with soluble anti-CD3. Error bars indicate s.e.m. IFN-γ production was measured by cytometric bead array (CBA). Similar results were obtained in 3 independent assays.

FIG. 4A shows the frequency of CD11b+ cells in naïve wild type and Tim-3 Tg mice, (p=0.0159, Mann-Whitney U test).

FIG. 4B shows the F480 expression on CD11b+ cells in naïve mice.

FIG. 4C shows Gr-1 expression on CD11b+ cells in naïve mice,

FIG. 5A shows the effect of sorted CD11b+ cells (5×10⁴/well) from either Tim-3 Tg mice or wild type littermates on the cell proliferation of wild type CD4+ T cells (5×10⁴/well) when stimulated with soluble anti-CD3 (0.5 μg/ml). Error bars indicated s.e.m.

FIG. 5B shows the effects of sorted CD11b+ cells (5×10⁴/well) from either Tim-3 Tg mice or wild type littermates on IFN-γ production by wild type CD4+ T cells (5×10⁴/well) when stimulated with soluble anti-CD3 (0.5 μg/ml). IFN-γ production was measured by cytometric bead array (CBA).

FIG. 6A shows the frequency of CD11b+ cells in Tim-3 Tg+ (n=7) and Tim-3 Tg− (n=8) RAG−/− recipients (p=0.0012, Mann-Whitney U test).

FIG. 6B shows the ratio of CD11b+F480^{low}Ly6G^{hi} to CD11b+F480^{hi}Ly6G¹′ cells in Tg+ (n=7) and Tg− (n=8) recipients (p=0.0059, Mann-Whitney U test).

FIG. 7 shows the effects of CD11b+F480^{low}Ly6G^{hi} and CD11b+ F480^{hi}Ly6G^{low} cells sorted from Tim-3 Tg+recipients that was used (5×10⁴/well) to stimulate wild type CD4+ T cells (5×10⁴/well) in the presence of soluble anti-CD3 (0.5 μg/ml). Error bars represent s.e.m. Similar results were obtained in an independent experiment.

FIG. 8A shows the cell proliferation and IFN-γ production by splenocytes (5×10⁵/well) from naïve Gal-9 Tg mice or wild type littermates when activated with soluble anti-CD3. Cell proliferation is measured by [³H]-thymidine incorporation. Cytokine production was determined by ELISA. Data shown are representative of eight experiments. (*p=0.007, Mann-Whitney U test).

FIG. 8B shows the cytokine IL-10 and IL-4 production by splenocytes (5×10⁵/well) from naïve Gal-9 Tg mice or wild type littermates when activated with soluble anti-CD3. Cytokine production was determined by ELISA. Data shown are representative of eight experiments. (**p=0.02, Mann-Whitney U test).

FIG. 9A shows the cell proliferation of cultured lymph node cells (5×10⁵/well) from TNP-OVA immunized Gal-9 Tg mice or wild type littermates (*p=0.001, Mann-Whitney U test). Data shown are representative of 4 experiments.

FIG. 9B shows the IFN-γ production by cultured lymph node cells (5×10⁵/well) from TNP-OVA immunized Gal-9 Tg mice or wild type littermates (**p=0.006, Mann-Whitney U test). Data shown are representative of 4 experiments.

FIG. 10A shows the CD62L expression in CD4+ lymph node cells from TNP-OVA immunized Gal-9 Tg mice or wild type littermates.

FIG. 10B shows the percentage of CD62Low expression in CD4+ lymph node cells from TNP-OVA immunized Gal-9 Tg mice or wild type littermates (*p=0.004, Mann-Whitney U test).

FIG. 11A shows the frequency of CD11b+ CD11c− cells in naïve Gal-9 Tg mice or wild type littermates (*p=0.0006, Mann-Whitney U test).

FIG. 11B shows the ratio of CD11b+Ly-6G+ to CD11b+Ly-6G− cells in naïve Gal-9 Tg mice or wild type littermates. (**p=0.03, Mann-Whitney U test).

FIG. 12A shows the effects of CD11b+ cells (10⁵/well) from either wild type or galectin-9 Tg mice on the cell proliferation and IFN-γ production by wild type CD4+ cells (10⁵/well) when stimulated with anti-CD3. Cell proliferation is measured by [³H]-thymidine incorporation. Cytokine production was determined by ELISA. (*p=0.02; **p=0.01, Mann-Whitney U test). Data shown are representative of four experiments.

FIG. 12B shows the effects of CD11b+ cells (10⁵/well) from either wild type or galectin-9 Tg mice on IL-10 and IL-4 production by wild type CD4+ cells (10⁵/well) when stimulated with anti-CD3. Cytokine production was determined by ELISA. (***p=0.04, Mann-Whitney U test; n=4)

FIG. 13A shows the frequency of CD11b+ cells in naïve wild type, Gal-9 Tg, or Gal-9 Tg×Tim-3^{-/-} mice (*p=0.02;**p=0.03; Mann-Whitney U test).

FIG. 13B shows the ratio of CD11b+Ly-6G+ to CD11b+Ly-6G−cells in naïve wild type, Gal-9 Tg, or Gal-9 Tg×Tim-3^{-/-} mice. (***p=0.03; ****p=0.03, Mann-Whitney U test).

FIG. 14A shows the effects of purified CD11b+ cells from wild type, Gal-9 Tg or Gal-9 Tg×Tim-3^{-/-} mice on the cell proliferation by wild type CD4+ (10⁵/well) when stimulated with anti-CD3. (*p=0.006, Mann-Whitney U test; n=5).

FIG. 14B shows the effects of purified CD11b+ cells from wild type, Gal-9 Tg or Gal-9 Tg×Tim-3^{-/-} mice on IFN-γ production by wild type CD4+ (10⁵/well) when stimulated with anti-CD3. IFN-γ production was measured by CBA. Data shown are representative of 5 experiments. (n=5).

FIG. 15A shows the cell proliferation of cultured lymph node cells (5×10⁵/well) from TNP-OVA immunized wild type, Gal-9 Tg or Gal-9 Tg×Tim-3^{-/-} mice upon re-activated with TNP-OVA. (*p=0.001, Mann-Whitney U test).

FIG. 15B shows the IFN-γ production by cultured lymph node cells (5×10⁵/well) from TNP-OVA immunized wild type, Gal-9 Tg or Gal-9 Tg×Tim-3^{-/-} mice upon re-activated with TNP-OVA. IFN-γ production was measured by CBA.

FIG. 16 shows the CD62L expression in CD4+ lymph node cells from immunized wild type, Gal-9 Tg or Gal-9 Tg×Tim-3^{-/-} mice.

FIG. 17 shows the relative expression of Galectin-9 expression in CD11b+Ly6G^{low} and CD11b+Ly6G^{hi} cells. CD11b+CD11c-Ly6G^{hi} and Ly6G^{low} cells were isolated from collagenase-digested wild type Balb/c spleens by cell sorting. Data shown are ΔΔCt where the expression of galectin-9 in CD11b+Ly6G^{low} cells was used as a calibrator. Error bars indicate s.e.m. The mean of two independent experiments is shown.

FIG. 18A shows the mean clinical disease score for the development of EAE in Tim-3 Tg (n=4) and wild type littermates (n=4) were immunized with 100 μg of MOG 35-55 in CFA and administered 100 ng pertussis toxin intravenously on Days 0 and 2. Error bars indicate SEM. n=3. Cummulative data are presented in Table 1.

FIG. 18B shows the linear regression curve for mean clinical disease score for the development of EAE in Tim-3 Tg (n=4) and wild type littermates (n=4) of FIG. 18A. The slopes are significantly different (p<0.0001). The 95% confidence interval for each curve is represented with dashed lines.

FIG. 18C shows that the Ly-6G (1A8) and F4/80 expression on CD11b+CD45hi cells from the CNS of a Tim-3 Tg mouse are free of disease (clinical score=0; open histogram) and a wild type mouse with disease (clinical score=3; shaded histogram).

FIG. 18D shows the frequency of F4/80lowLy-6G+, Ly-6G+ and F4/80low cells in the CD11b+CD45hi population from the CNS of Tim-3 Tg and wild type mice with disease (clinical score=3) and without disease (clinical score=0). Error bars indicate SEM. (*p=0.0028, **p=0.0062, ***p=0.0002, unpaired t-test compared to wild type with disease).

### DETAILED DESCRIPTION OF THE INVENTION

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Molecular Biology, Vol. 203, 2003, Transgenic Mouse, editored by Marten H. Hofker and Jan van Deursen, which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean ±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention relates to methods of modulating the expansion myeloid-derived suppressor cells (MDSCs) and uses thereof for the purpose of modulating the immune system.

Embodiments of the present invention are based on the surprising discovery that the activation of the Tim-3/galectin-9 signaling pathway is involved in the in vivo expansion of a specific population of immature immune cells known as myeloid-derived suppressor cells (MDSCs). While not wishing to be bound by theory, it is believed that the Tim-3 pathway in T-cells is activated by the direct interaction of Tim-3 with its known natural ligand, galectin-9, which results in the tyrosine phosphorylation of the cytoplasmic portion of the transmembrane Tim-3 receptor protein. Moreover, the Tim-3:galectin-9 interaction was necessary for the expansion of MDSCs.

Tim-3 is a member of the recently discovered Tim family (T cell Immunoglobulin and mucin domain) that is specifically expressed on IFN-γ-secreting Th1 cells and is also constitutively expressed on dendritic cells, but not on peripheral macrophages ¹¹. Tim-3 and its known ligand, galectin-9, regulate Th1 immunity by inducing cell death in IFN-γ secreting Th1 cells ¹². Tim-3 also influences immune tolerance ^{13,14}.

Galectin 9, which is a physiologically active substance acting as a lectin, is expressed in tissue mast cells, eosinophils, macrophages, T cells, B cells, fibroblasts, endothelial cells, various tumor cells and other cells.

Early studies using Tim3-specific monoclonal antibodies indicated that Tim-3 acts as a negative regulator of autoimmune responses. Administration of Tim-3-immunoglobulin fusion proteins during a Th1-cell-mediated immune response led to hyperproliferation of Th1 cells and increased production of Th1-type cytokines, indicating that Tim3 normally acts to inhibit Th1-cell effector responses. Sánchez-Fueyo et al. studied the effects of blocking the Tim-3 pathway in NOD mice (which spontaneously develop insulin-dependent diabetes, an autoimmune disease). Treatment with Tim-3-specific monoclonal antibodies accelerated the onset of disease in these mice, confirming earlier reports of a role for this protein in inhibiting autoimmune responses.

Studies have implicated IFN-γ in MDSC-mediated immune suppression. Tim-3 is a cell surface receptor expressed on Th1 cells and is involved in the negative regulation of IFN-γ secreting cells. The inventors now show that the Tim-3/galectin-9 signaling pathway plays a role in the expansion and/or activation of MDSCs. The inventors show, using Tim-3 overexpressing transgenic mice, that the Tim-3 pathway-dependent MDSCs expansion can be greatly enhanced by the overexpression of Tim-3 in T-cells. While not wishing to be bound by theory, more Tim-3 receptors on T-cells can shift the in vivo physiological binding equilibrium between Tim-3 and galectin-9 towards Tim-3 since there could be more Tim-3 available to bind the limited amount of ligand galectin-9. The increased Tim-3/galentin-9 interaction leads to increased activation of the Tim-3 pathway and consequently enhanced expansion of MDSCs.

The inventors also show, using galectin-9 overexpressing transgenic mice, that the Tim-3 pathway-dependent MDSCs expansion can be greatly enhanced by the overexpression of galectin-9. While not wishing to be bound by theory, excess circulating galectin-9 can also shift the in vivo physiological binding equilibrium between Tim-3 and galectin-9 towards Tim-3 since there could then be an excess of galectin-9 available to saturate a greater number of Tim-3 receptors on T cells. It is believed that the increased Tim-3/galectin-9 interaction leads to increased activation of the Tim-3 pathway and consequently enhanced expansion of MDSCs.

The inventors also show that the Tim-3:galectin-9 interaction was necessary for the expansion of MDSCs. The overexpression of galectin-9 in Tim-3−/− deficient mice did not lead to an expansion of the MDSCs. While not wishing to be bound by theory, it is thought that the Tim-3:galectin-9 interaction initiates the Tim-3 signaling events intracellularly in the T-cell. These intracellular signaling events eventually result in the expression of effector molecules that can directly or indirectly promote the expansion of MDSCs. Examples of effector molecules include but are not limited to proteins and/or peptide ligands for a surface receptor on the MDSCs. Such a ligand can be secreted or bound to the surface of the T-cell.

Alternatively, or in addition, while not wishing to be bound by theory, the Tim-3:galectin-9 interaction-dependent intracellular signaling events in the T-cell could elicit a cell-to-cell interaction between the T-cell and MDSCs that leads to the proliferation of the MDSC population. A cell-to-cell interaction can be, for example, via the interaction of a newly expressed cell-surface ligand on T-cells with a cell surface receptor on the MDSCs. In this alternative, the expression of the cell-surface ligand on T-cells is dependent on the Tim-3:galectin-9 interaction and subsequent Tim-3 signaling pathway. Another example of a cell-to-cell interaction could be via the interaction of the Tim-3 on a T-cell with galectin-9 and an associated cell surface receptor on the MDSCs. It is contemplated that the galectin-9/Tim-3 complex undergoes modification that is dependent on the Tim-3:galectin-9 interaction and subsequent Tim-3 signaling pathway. The modification of the Tim-3/galectin-9 complex elicits signaling events through the galectin-9 associated cell surface receptor on MDSCs to bring about the expansion of the MDSC population. Such cell-to-cell interactions between T-cells and MDSCs mediate cellular signals within the MDSCs, eventually resulting cell proliferation of MDSCs.

The inventors also discovered high expression of galectin-9 on the MDSCs. While not wishing to be bound by theory, it is contemplated that the interaction of the Tim-3 on T-cells with the galectin-9 on MDSCs induces cell expansion of MDSCs. Such interaction between Tim-3 and galectin-9 enables the interaction of T-cells with MDSCs. In one aspect, the Tim-3:galectin-9 interaction in this case initiates the Tim-3 signaling pathway in T-cells as well as a cell expansion signal cascade within the MDSCs. In another aspect, the Tim-3 signaling pathway elicited a cell expansion signal to the MDSCs. The cell expansion signal can be a newly expressed ligand for a cell surface receptor on MDSCs. In yet another aspect, Tim-3 signaling pathway in T-cells initiates a modification of the galectin-9/Tim-3 complex, resulting in a transduction of an cell expansion signal into MDSCs via the attached galectin-9. In yet another aspect, Tim-3 acts as a ligand for galectin-9 and an associated membrane protein on MDSCs, triggering a cell expansion signal into MDSCs.

When in vivo MDSCs are greatly expanded, the mice exhibit reduced ability to mount an immune response when challenged with anti-CD3 antibodies. In the mouse model of multiple sclerosis, Experimental Autoimmune Encephalomyelitis (EAE), mice with a larger population of MDSCs failed to exhibit the classic physiological and histological pathologies associated with the disease. Hence, the immune system is suppressed by a larger population of MDSCs in vivo.

Accordingly, embodiments of the invention provide methods for modulating a population of MDSCs in a subject in need of such modulation, the method comprising administering to the subject a therapeutically effective amount of an agent that modulates the Tim-3 pathway.

As used herein, the term “modulating a population of MDSCs” refers to an increase or decrease in the number of MDSCs relative to a subject not treated with an agent that modulates the population of MDSCs. An “increase” or “decrease” refers to a statistically significant increase or decrease respectively. For the avoidance of doubt, an increase or decrease will be at least 10% relative to a reference, preferably at least 10%, 20%, 30%, 40%, 50%, 60%, 70%, 80%, 90%, or more, up to and including at least 100% or more, in the case of an increase, for example, at least 2×, 3×, 4×, 5×, . . . 10× or more). All the percentages in between 5-100% as well as fractions of integer number of folds increase are also included.

As used herein, subjects who are in need of modulation of a population of MDSCs include but are not limited cancer treatment patients, an individual diagnosed with an autoimmune disease, an immunocompromised individual, organ transplant recipients, and individuals suffering from infections by pathogens.

In addition, subjects can include individuals who are at high risk of acquiring infections, for example, individuals who are taking drugs that suppress the immune system, such as anticancer drugs (chemotherapy), corticosteroids and other immunosuppressant drugs. Individuals with certain diseases and conditions are also susceptible to infections by pathogens, for example, individuals suffering from HIV/AIDS, kidney failure, diabetes, lung disease, such as emphysema, Hodgkin's disease or other lymphomas, leukemia, and extensive burns. Such individuals can also be subjects in need of treatment as described herein.

In one embodiment, the autoimmune diseases include but are not limited to rheumatoid arthritis, multiple sclerosis, inflammatory bowel diseases, myasthenia gravis, lupus erythematosus, Guillain-Barré syndrome, Grave's disease, Diabetes mellitus type 1, celiac disease, Addison's disease, and autoimmune hepatitis.

In one embodiment, an immunocompromised individual suffers from HIV or is undergoing anti-cancer treatment.

In one embodiment, pathogens that can caused infections include bacteria, viruses, fungi, and parasites. Pathogens can include, for example, Clostridium botulism, Clostridium difficile, Bordetella pertussis, Listeria monocytogenes, Neisseria meningitides, Haemophilus influenzae, Brucella species, Coxiella burnetii, Shigella species, Escherichia coli O157:H7, Mycoplasma pneumoniae, Mycoplasma tuberculosis, Mycoplasma avium-intracellular complex, Mycoplasma gordonae, Mycoplasma kansaii, Staphylococci aurenus, Staphylococci epidermidis, Staphylococci saprophiticus, Staphylococci lugdunensis, Streptococcus pyogenes, Streptococcus agalactiae, Streptococcus pneumoniae Enterococcus faecalis, Enterococcus faecium, Enterococcus gallinarum, Enterococcus casseliflavus, Klebsiella pneumoniae, Klebsiella oxytoca, Pseudomonas aeruginosa, Acinetobacter baumannii, Nocardia species, Salmonella species, Vibrio species, and Yersinia, Human Immuno deficiency Virus (HIV), influenza, severe acute respiratory syndrome coronavirus, (SARS-CoV), hepatitis B virus, hepatitis C virus, respiratory syncytial virus, herpes simplex virus, Candida species, Aspergillus species, Fasciola hepatica, Paragonimus westermani, Loa boa, Clonorchis sinensis, Echinococcus species, Dirofilaria species, Hymenolepis nana, Strongyloides stercoralis, Trypanosoma brucei gambiense (West African), Trypanosoma brucei rhodesiense (East African), Trypanosoma cruzi, Wuchereria bancrofti, Brugia malayi, Brugia timori, and Giardia lamblia.

As used herein, the term “a therapeutically effective amount” refers an amount sufficient to achieve the intended purpose. For example, an effective amount of an agent that activates the Tim-3 pathway will cause an expansion of MDSCs as the term defined herein and consequently suppress the immune system. An effective amount of an agent that inhibits the Tim-3 pathway will suppress the expansion of MDSCs as the term defined herein and consequently upregulate and activate of the immune system. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

As used herein, the term “an agent” refers to any inorganic or organic molecule or compound that modulate the Tim-3 signaling pathway. An agent can bind to Tim-3, such as to the extracellular region of Tim-3. The binding can be inhibitory or activating. The binding of Tim-3 with an inhibitory agent will not result in any signal transduction intracellularly by the Tim-3. On the other hand, binding of Tim-3 to an activating agent will elicit signal transduction in the cell. An agent can also induce or inhibit signal transduction by Tim-3 without direct binding to Tim-3. An agent can also change the expression of Tim-3 or galectin-9.

In one embodiment, an agent can activate the Tim-3 pathway, whereby there is an increase in the tyrosine phosphorylation of the cytoplasmic portion of Tim-3 and downstream signaling events are elicited. The changes in the tyrosine phosphorylation of Tim-3 can be assayed by methods known in the art, such as the immunoprecipitation of Tim-3 by antibodies specific for Tim-3 followed by a Western blot analysis for phosphor-tyrosine (pY) using antibodies specific for pY. The data obtained for an activating agent can be compared to the data obtained for a control agent that does not activate the Tim-3 pathway or to data obtained in the absence of any added agent. The latter two data are control data. An increase of pY that is at least 10% more relative to the control, preferably at least 20%, 30%, 40%, 50%, 60% 70%, 80%, 90%, or more, up to and including at least 100% or more greater pY compared to the control is expected. Examples of agents that activate the Tim-3 pathway include but are not limited to Tim-3-binding molecules or compounds, galectin-9, galectin-9 analogs, fragments, and derivatives, galectin-9-peptido-mimetics and activating Tim-3 antibodies.

In another embodiment, an agent can inhibit the Tim-3 pathway whereby no increase in tyrosine phosphorylation of the cytoplasmic portion of Tim-3 occurs and/or downstream signaling events are not elicited. When the Tim-3 pathway is inhibited, one or more of the intracellular signaling events of Tim-3 post-activation are blocked. It is expected that there is no increase in the level of the tyrosine phosphorylation of Tim-3. Examples of inhibiting agents include but are not limited to inhibiting Tim-3 antibodies that bind to the extracellular regions of Tim-3, soluble Tim-3 proteins or conjugates, galectin-9-binding molecule or protein or antibody, carbohydrate ligands or mimetics that bind the carbohydrate recognition domain (CRD) of galectin-9, small interfering RNA that is specific for or target to Tim-3 or galectin-9, and antisense RNA that hybridize specifically to with the messenger RNA of Tim-3 or galectin-9. In one embodiment, galectin-9, anti-sense nucleic acid to Tim-3, anti-sense nucleic acid to galectin-9, soluble Tim-3 and conjugates thereof, and inhibiting Tim-3 antibodies from hybridomas 8B.2C12 and 25F.1D6 are prepared as disclosed in U.S. Patent application Nos: 2004-0005322 and 2005-0191721, Sabatos, C. A. et al., Nature Immunol. 4:1102-1110, 2003, and Sanchez-Fueyo, A. et al., Nature Immunol. 4:1093-101 2003, which are hereby incorporated by reference in their entirety.

As used herein, the term “galectin-9 analogs, fragments or derivatives” refer to a galectin-9 that is encoded by a variant galectin-9 polynucleotide coding sequence or a galectin-9 that is a splice variant that has resulted from the post-transcriptional processing of the galectin-9 primary transcript. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the galectin-9 protein or portions thereof. Also especially preferred in this regard are conservative substitutions.

Galectin-9 derivatives are galactin-9 that are expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, hIL5-receptor has been fused with Fc portions for the purpose of high-throughput screening assays to identify antagonists of hIL-5. See, D. Bennett et al., Journal of Molecular Recognition, Vol. 8 52-58 (1995) and K. Johanson et al., J. Biol. Chem., Vol. 270, 16:9459-9471 (1995).

In one embodiment, galectin-9, galectin-9 analogs, fragments, and derivatives are made according to methods disclosed in U.S. Pat. No. 6,468,768 and U.S. Patent application No. 2005-0191721. Lactose binding activity of the expressed galectin-9 can be assayed by immunodetection of in situ binding activity to asialofetuin immobilized on nitrocellulose (Madsen et al., J. Biol. Chem. 270(11):5823-5829 (1995)). Thirty μg of asialofetuin dissolved in 3 μl of water is spotted on a 1-cm2 strip of nitrocellulose. The nitrocellulose pieces are then placed in a 24-well tissue culture plate and incubated overnight in buffer B (58 mM Na₂ HPO₄, 18 mM KH₂ PO₄, 75 mM NaCl, 2 mM EDTA, and 3% BSA, pH7.2) with constant agitation at 22° C. Following incubation, the blocking medium is aspirated and the nitrocellulose pieces are washed three times in buffer A (58 mM Na₂ HPO₄, 18 mM KH₂ PO₄, 75 mM NaCl, 2 mM EDTA, 4 mM β-mercaptoethanol and 0.2% BSA, pH 7.2). Cell extracts (preferably, COS cells) are prepared containing 1% BSA and either with or without 150 mM lactose (105 μl of primary extract, 15 μl of 10% BSA in buffer A and either 30 μl of 0.75 M lactose in buffer A or 30 μl of buffer A). The immobilized asialofetuin is incubated with the extracts for 2 h and washed 5 times in buffer A. The nitrocellulose pieces are then fixed in 2% formalin in PBS (58 mM Na₂ HPO₄, 18 mM KH₂ PO₄, 75 mM NaCl, 2 mM EDTA pH7.2) for 1 hour to prevent loss of bound galectin. Following extensive washing in PBS the pieces were incubated with rabbit anti-galectin 9 polyclonal serum diluted 1:100 in PBS for 2 h at 22° C. The pieces are then washed in PBS and incubated with peroxidase-labeled goat anti-rabbit antibodies (DAKO). Following incubation for 2 h at 22° C., the pieces are washed in PBS and the substrate is added. Nitrocellulose pieces are incubated until the color develops and the reaction is stopped by washing in distilled water.

In one embodiment, Tim-3 can be made according to methods disclosed in Monney et. al., Nature 415: 536-541 (2002), Anderson, A., et. al., Science, 318:1141-3 (2007), and U.S. Patent application Nos. 2004-0005322 and 2005-0191721.

In another embodiment, Tim-3 or galectin-9 can be cloned and expressed according to molecular methods known to one skilled in the art. In one embodiment, the soluble Tim-3 can be expressed as a fusion protein or conjugated to an antibody Fc fragment. In yet another embodiment, the gene encoding Tim-3 or galectin-9 can be cloned into an expression vector for the overexpression of the respective protein.

Examples of expression vectors and host cells are the pET vectors (NOVAGEN®), pGEX vectors (GE Life Sciences), and pMAL vectors (New England labs. Inc.) for protein expression in E. coli host cell such as BL21, BL21(DE3) and AD494(DE3)pLysS, Rosetta (DE3), and Origami(DE3) ((NOVAGEN®); the strong CMV promoter-based pcDNA3.1 (INVITROGEN™ Inc.) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech), pAd/CMV/V5-DEST, pAd-DEST vector (INVITROGEN™ Inc.) for adenovirus-mediated gene transfer and expression in mammalian cells; pLNCX2, pLXSN, and pLAPSN retrovirus vectors for use with the RETRO-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells; BACpak6 baculovirus (Clontech) and pFastBac™ HT (INVITROGEN™ Inc.) for the expression in Spodopera frugiperda 9 (Sf9) and Sf11 insect cell lines; pMT/BiP/V5-His (INVITROGEN™ Inc.) for the expression in Drosophila Schneider S2 cells; Pichia expression vectors pPICZα, pPICZ, pFLDα and pFLD (INVITROGEN™ Inc.) for expression in Pichia pastoris and vectors pMETα and pMET for expression in P. methanolica; pYES2/GS and pYD1 (INVITROGEN™ Inc.) vectors for expression in yeast Saccharomyces cerevisiae. Recent advances in the large scale expression heterologous proteins in Chlamydomonas reinhardtii are described by Griesbeck C. et. al. 2006 Mol. Biotechnol. 34:213-33 and Fuhrmann M. 2004, Methods Mol. Med. 94:191-5. Foreign heterologous coding sequences are inserted into the genome of the nucleus, chloroplast and mitochodria by homologous recombination. The chloroplast expression vector p64 carrying the most versatile chloroplast selectable marker aminoglycoside adenyl transferase (aadA), which confer resistance to spectinomycin or streptomycin, can be used to express foreign protein in the chloroplast. Biolistic gene gun method is used to introduced the vector in the algae. Upon its entry into chloroplasts, the foreign DNA is released from the gene gun particles and integrates into the chloroplast genome through homologous recombination.

In one embodiment, the expression vector is a viral vector, such as a lentivirus, adenovirus, or adeno-associated virus. A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of Stratagene's ADEASY™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In one embodiment, the preferred viral vector is a lentiviral vector and there are many examples of use of lentiviral vectors for gene therapy for inherited disorders of haematopoietic cells and various types of cancer, and these references are hereby incorporated by reference (Klein, C. and Baum, C. (2004). Hematol. J., 5, 103-111; Zufferey, R et. al. (1997). Nat. Biotechnol., 15, 871-875; Morizono, K. et. al. (2005). Nat. Med., 11, 346-352; Di Domenico, C. et. al. (2005). Gene therapy for amucopolysaccharidosis type I murine model with lentiviral-IDUA vector. Hum. Gene Ther., 16, 81-90). The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with VIRAPOWER™ Lentiviral Expression systems from Invitrogen.

In one embodiment, the expression viral vector can be a recombinant adeno-associated virus (rAAV) vector. Using rAAV vectors, genes can be delivered into a wide range of host cells including many different human and non-human cell lines or tissues. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the chimeric DNA coding sequence, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

As used herein, the term “antibody” refers to immunoglobulin molecules and immunologically active portions of immunoglobulin molecules, i.e., molecules that contain an antigen binding site that immunospecifically binds an antigen. The terms also refers to antibodies comprised of two immunoglobulin heavy chains and two immunoglobulin light chains as well as a variety of forms besides antibodies; including, for example, Fv, Fab, and F(ab)′₂ as well as bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2nd ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference.).

In one embodiment, antibodies made against Tim-3 or galectin-9 are synthesized according to methods disclosed in Monney et. al., (2002). In another embodiment, recombinant Tim-3 or galectin-9 protein or fragments thereof can be use for immunization. Methods for induce antibodies according to methods well known in the art including, but not limited to, in vivo immunization, in vitro immunization, and phage display methods. See, e.g. Wilson et al., Cell 37:767 (1984) and Bittle et al., J. Gen. Virol., 66:2347-2354 (1985). If in vivo immunization is used, animals can be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde.

In one embodiment, the Tim-3 or galectin-9 antibody is a polyclonal antibody. In one embodiment, the Tim-3 or galectin-9 antibody is a monoclonal antibody. In preferred embodiment, the Tim-3 or galectin-9 antibody is a humanized antibody. In preferred another embodiment the Tim-3 or galectin-9 antibody is a chimeric antibody. In yet another embodiment, the Tim-3 or galectin-9 antibodies include, but are not limited to multispecific, human, single chain antibodies, Fab fragments, F(ab)′2 fragments, fragments produced by a Fab expression library, domain-deleted antibodies (including, e.g., CH2 domain-deleted antibodies), anti-idiotypic (anti-Id) antibodies (including, e.g., anti-Id antibodies to antibodies of the invention), and epitope-binding fragments of any of the above. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Encompassed in the methods disclosed herein are Tim-3 or galectin-9 antibodies that are, but are not limited to, engineered forms of antibodies and antibody fragments such as diabodies, triabodies, tetrabodies, and higher multimers of scFvs, single-domain antibodies, as well as minibodies, such as two scFv fragments joined by two constant (C) domains. See, e.g., Hudson, P. J. and Couriau, C., Nature Med. 9: 129-134 (2003); U.S. Publication No. 20030148409; U.S. Pat. No. 5,837,242.

In one embodiment, the Tim-3 or galectin-9 antibodies can be obtained from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

In a preferred embodiment for use in humans, the Tim-3 or galectin-9 antibodies are human or humanized antigen-binding antibody fragments of the present invention and include, but are not limited to, Fab, Fab and F(ab)′₂, Fd, single-chain Fvs (scFv), single-domain antibodies, triabodies, tetrabodies, minibodies, domain-deleted antibodies, single-chain antibodies, disulfide-linked Fvs (sdFv) and fragments comprising either a variable light chain (VL) or variable heavy chain VH region. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains.

Preferred antibodies in the therapeutic methods of the invention are those containing a deletion of the CH2 domain.

As used herein, the term “humanized” immunoglobulin or “humanized” antibody refers to an immunoglobulin comprising a human framework, at least one complementarity determining regions (CDR) from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. For example, a humanized immunoglobulin would not encompass a chimeric mouse variable region/human constant region antibody.

As used herein, the term “framework region” refers to those portions of antibody light and heavy chain variable regions that are relatively conserved (i.e., other than the CDRs) among different immunoglobulins in a single species, as defined by Kabat, et al., op. cit. As used herein, a “human framework region” is a framework region that is substantially identical (about 85% or more) to the framework region of a naturally occurring human antibody.

As used herein, the term “chimeric” antibody refers to an antibody whose heavy and light chains have been constructed, typically by genetic engineering, from immunoglobulin gene segments belonging to different species. For example, the variable (V) segments of the genes from a mouse monoclonal antibody may be joined to human constant (C) segments, such as gamma1 and/or gamma4. A typical therapeutic or diagnostic chimeric antibody is thus a hybrid protein comprising at least one V region (e.g., VH or VL) or the entire antigen-binding domain (i.e., VH and VL) from a mouse antibody and at least one C (effector) region (e.g., CH (CH1, CH2, CH3, or CH4) or CL or the entire C domain (i.e., CH and CL) from a human antibody, although other mammalian species may be used. In some embodiments, especially for use in the therapeutic methods of the Tim-3 or galectin-9 antibodies should contain no CH2 domain.

In one embodiment, a chimeric antibody may contain at least the Tim-3 or galectin-9 antigen binding Fab or F(ab)′₂ region while the humanized antibody can contain at least the Tim-3 or galectin-9 antigen binding Fv region fused to a human Fc region.

The terms “antigen” is well understood in the art and refer to the portion of a macromolecule which is specifically recognized by a component of the immune system, e.g., an antibody or a T-cell antigen receptor. The term antigen includes any protein determinant capable of specific binding to an immunoglobulin. Antigenic determinants usually consist of chemically active surface groupings of molecules such as amino acids or sugar side chains and usually have specific three dimensional structural characteristics, as well as specific charge characteristics.

In one embodiments, the Tim-3 antibodies (including molecules comprising, or alternatively consisting of, antibody fragments or functional variants thereof), immunospecifically bind to the extracellular portion of Tim-3 and do not cross-react with any other antigens. In one embodiments, the galectin-9 antibodies (including molecules comprising, or alternatively consisting of, antibody fragments or functional variants thereof), immunospecifically bind to galectin-9 and do not cross-react with any other antigens.

The term “functional variants” as used herein refers to the antibody or fragments thereof that have amino acids mutations in the protein. The mutations results in comparable or greater Tim-3 or galectin-9 binding of the parent antibody protein.

In one embodiment, the Tim-3 antibodies disclosed herein (including molecules comprising, or alternatively consisting of, antibody fragments or functional variants thereof) preferentially bind the extracellular portion of Tim-3, or fragments thereof relative to their ability to bind other antigens. In one embodiment, the galectin-9 antibodies of the invention (including molecules comprising, or alternatively consisting of, antibody fragments or functional variants thereof) preferentially bind galectin-9, or fragments thereof relative to their ability to bind other antigens.

In one embodiment, a hybridoma cell line comprising a nucleic acid molecule encoding a Tim-3 or galectin-9 antibody can be made. Naïve BALB/c mice are immunized with Tim-3 or galectin-9 or fragments thereof in complete Freund's adjuvant. Alternatively, a transgenic animal that has been genetically modified to produce human antibodies, such as XENOMOUSE™ and HuMab mouse, or a transchromosome (TC) mouse, may be immunized to generate the polyclonal antibodies. Hybridoma cell lines, specific for Tim-3 or galectin-9, can be prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 571-681 (1981)). Briefly, hybridoma cell lines were generated using standard PEG fusion of the non-secreting myeloma cells (P3x63.Ag8) to splenocytes overexpressing Tim-3 or galectin-9 antibodies at a ratio 1:3 and selected in Hat (hypoxanthin, aminopterin, and thymidine) media in 96 well plates. After two weeks, individual supernatants were tested for reactivity with anti-Tim-3 or galectin-9 activity by ELISA, Western blot, and immunohistochemistry. Positive hybridomas colonies were subcloned and screened for reactivity twice to ensure clonality. Antibodies were isolated from hybridoma supernatants by protein G affinity purification using standard methods.

From these hybridoma cell lines producing the specific Tim-3 or galectin-9 antibodies, the polynucleotides encoding the VL and VH regions of these antibodies can be cloned into cloning vectors such as TOPO® vectors (INVITROGEN™ Inc.) and used for further molecular biology manipulations to generate other chimeric and humanized antibodies, variant forms of Tim-3 or galectin-9 antibodies, and/or recombinant Tim-3-binding or galectin-9-binding proteins.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

In one embodiment, the Tim-3 or galectin-9 antibodies can be produced by any method known in the art for the synthesis of antibodies, in particular, by chemical synthesis or preferably, by recombinant expression techniques.

Recombinant expression of an antibody disclosed herein, or fragment, derivative or analog thereof, (e.g., a heavy or light chain of an antibody of the invention or a single chain antibody of the invention), including a recombinant protein derived from the antibody antigen-binding region, requires construction of an expression vector containing a polynucleotide that encodes the antibody. Once a polynucleotide encoding an antibody molecule or a heavy or light chain of an antibody or portion thereof (preferably containing the heavy or light chain variable domain) of the invention has been obtained, the vector for the production of the antibody molecule may be produced by recombinant DNA technology using techniques well known in the art. Thus, methods for preparing a protein by expressing a polynucleotide containing an antibody-encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT publication WO 86/05807; PCT publication WO 89/01036; and U.S. Pat. No. 5,122,464) and the variable domain of the antibody may be cloned into such a vector for expression of the entire heavy or light chain. Methods for generating multivalent and bispecific antibody fragments are described by Tomlinson I. and Holliger P. (2000) Methods Enzymol, 326, 461-479 and the engineering of antibody fragments and the rise of single-domain antibodies is described by Holliger P. (2005) Nat. Biotechnol. September; 23(9):1126-36, and are both hereby incorporated by reference.

In one embodiment, agents that affect the expression levels of Tim-3 or galectin-9 can be small interfering nucleic acids to coding gene of Tim-3 or galectin-9, or an anti-sense nucleic acid to Tim-3 or galectin-9 capable of hybridizing with a nucleic acid encoding Tim-3 or galectin-9 under stringent hybridization conditions. Anti-sense nucleic acid to Tim-3 is prepared according to the method disclosed in U.S. Patent application No: 2004-0005322. Anti-sense nucleic acid to galectin-9 can be similarly designed with methods disclosed and those known in the art.

RNA interference-inducing molecules, for example but are not limited to siRNA, dsRNA, stRNA, shRNA, micro RNAi (mRNAi), antisense oligonucleotides etc. and modified versions thereof, where the RNA interference molecule silences the gene expression of Tim-3 or galectin-9. An anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent can be a cellular gene or genomic sequence, e.g. the Tim-3 (Genbank Accession No. AF450242, AF450243, DD240371, DD240372) or the galectin-9 (Genbank Accession No. NM_{—}002308 and NM_{—}009587) sequence. An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotidesmolecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

siRNAs useful for the methods described herein include siRNA molecules of about 15 to about 40 or about 15 to about 28 nucleotides in length, which are homologous to the Tim-3 or galectin-9 gene. Preferably, the Tim-3/galectin-9 targeting siRNA molecules have a length of about 19 to about 25 nucleotides. More preferably, the targeting siRNA molecules have a length of about 19, 20, 21, or 22 nucleotides. The targeting siRNA molecules can also comprise a 3′ hydroxyl group. The targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the Tim-3 or galectin-9 targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the targeting RNA molecule is double stranded—one strand has a 3′ overhang and the other strand can be blunt-ended or have an overhang. In the embodiment in which the targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

In one embodiment, the siRNA or modified siRNA is delivered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting Tim-3 or galectin-9. In one embodiment, the vector can be a regulatable vector, such as tetracycline inducible vector.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, retroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

Efficacy of the siRNA on the expression of Tim-3 or galectin-9 can be monitored using methods know in the art such as quantitative RT-PCR with specific oligonucleotide primers for each gene respectively, or ELISA for Tim-3 and/or galectin-9 from a sample of peripheral blood. Alternately, the population of MDSCs can be determined by FACS analysis using the markers characteristic of MDSCs as disclosed herein.

In another embodiment, the agent that modulate the Tim-3 pathway can be the coding nucleic acid for full-length Tim-3 or galectin-9, and is carried in a vector for transport and protein expression in living mammalian cells, for example, an adeno-associated virus. Upon transfection into a cell, the agent enables overexpression of either Tim-3 or galectin-9 in vivo. Cloning and over expression of the full-length Tim-3 and galectin-9 can be performed as disclosed in U.S. Patent application Nos: 2004-0005322 and 2005-0191721.

Envisioned in the methods disclosed herein are agents that inhibit, block, promote, and/or enhance the signaling events within the Tim-3 pathway.

As used herein, the term “Tim-3 pathway” refers to the phosphorylation of the cytoplasmic portion of the transmembrane Tim-3 receptor protein elicited by Tim-3 binding to a ligand, the other subsequent downstream signaling events on T-cells and/or MDSCs, and/or cell-to-cell interactions brought about be Tim-3:galectin-9 interaction and the consequential events that leads to the expansion of the MDSCs. The ligands that Tim-3 can bind include but are not limited to galectin-9, galectin-9 analogs, fragments, and derivatives, galectin-9-peptido-mimetics and activating Tim-3 antibodies. The galectin-9 can be soluble or bound on the surface of cells that express it. One hallmark of activation of the Tim-3 pathway is the phosphorylation of the intracellular domain of Tim-3. Thus, one of skilled in the art can determine whether a given agent modulates the Tim-3 pathway by monitoring the phosphorylation of the intracellular domain of Tim-3. Alternately, the number of MDSCs can be monitored, for example, by FACS analysis as described herein, using those markers that are characteristic of the MDSC population as described herein. The terms “Tim-3 pathway” and “Tim-3:galectin-9 signaling pathway” are used interchangeably herein.

In one embodiment, the Tim-3 pathway is activated by galectin-9. In another embodiment, the Tim-3 pathway is activated by a galectin-9 analog, fragment or derivative. In yet another embodiment, the Tim-3 pathway is activated by an activating Tim-3 antibody or fragments or conjugates thereof. An activating Tim-3 antibody is one which, upon binding to the extracellular portion of Tim-3, results in the tyrosine phosphorylation of the cytoplasmic portion of Tim-3 that is identical to that elicited when Tim-3 is bound to its known natural ligand, galectin-9. The protein phosphorylation of the cytoplasmic portion of Tim-3 that resulted from the binding of an activating Tim-3 antibody elicits identical downstream signaling cascade, galectin-9:Time-3 complex modification, gene expressions, and/or cell-to-cell interactions that leads to the expansion of the MDSCs.

As used herein, the term “MDSC” refers to a specialized population of cells that are of the hematopoietic lineage and express the macrophage/monocyte marker CD11b+ and the granulocyte marker Gr-1/Ly-6G. MDSCs express low or undetectable expression of the mature antigen presenting cell markers MHC Class II and F480. The MDSCs are immature cells of the myeloid lineage and can further differentiate into several cell types, including macrophages, neutrophils, dendritic cells, Langerhand cells, monocytes or granulocytes. MDSCs can be found naturally in normal adult bone marrow of human and animals or in sites of normal hematopoiesis, such as the spleen. MDSCs suppress T cell responses by various mechanisms including but are not limited to production of reactive oxygen species, peroxynitrites, increased arginase metabolism due to high levels of arginase, and increased nitrous oxide synthase. T cell responses include but are not limited to T-cell activation, T-cell proliferation, and T-cell cytokine production. For example, MDSCs can response to IFN-γ and several cytokines such as IL-4 and IL-13. IFN-γ can activate MDSCs which induces the activity of nitric-oxide synthase 2 (NOS2). Alternately, Th2 cytokines such as interleukin-4 (IL-4) and IL-13 can activate MDSCs which leads to the induction of arginase-1 (ARG1) activity. The metabolism of L-arginine by either NOS2 or ARG1 leads to the inhibition of T-cell proliferation, and the activity of both enzymes together can result in T-cell apoptosis through the production of reactive nitrogen-oxide species.

Mature macrophages do not express the granulocyte marker Gr-1/Ly-6G, but exhibit a high expression of F480. In a comparison of the expressions of Gr-1/Ly-6G and F480 on MDSCs and mature macrophages analyzed by flow cytometry using the same antibodies specific against Gr-1/Ly-6G or F480, and using the same fluorophores on these antibodies, the MDSCs exhibit at least 10% lower in the median fluorescence intensity obtained for F480 compared to that obtained for mature macrophage. The MDSCs can exhibit at least 10%, . . . at least 30%, . . . at least 50%, . . . at least 70%, . . . at least 100% (that is no detectable F480 fluorescence), including the percentages between 10% and 100%, lower median fluorescence intensity obtained for F480 compared to that of mature macrophage. On the other hand, MDSCs exhibit at least 50% higher in the median fluorescence intensity obtained for Gr-1/Ly-6G compared to that of mature macrophage. The MDSCs can exhibit at least 50% greater, for example, at least 100% greater, at least 500%, . . . at least 1000% and beyond, including the percentages between 50% and 1000%, higher median fluorescence intensity obtained for Gr-1/Ly-6G compared to that of mature macrophages.

In one embodiment, the MDSCs can be isolated as described in the International Patent Application PCT/US2007/060210 and Gallina, G, et. al, 2006, J. Clin, Invest. 116: 2777-2790. In another embodiment, the MDSCs can be isolated by methods known in the art, such as via cell sorting by flow cytometry using antibodies specific for the markers CD11b, Gr-1/Ly-6G and F480 as disclosed herein.

Accordingly, the method described herein promotes the expansion of MDSCs in a subject afflicted with an autoimmune disease or disorder, the method comprising administering a therapeutically effective amount of an agent that activates Tim-3 pathway. The potent immunosuppressive effects of the expanded MDSCs help in ameliorating the symptoms of the autoimmune disease or disorder.

In one embodiment, the method described herein can be administered in conjunction with other therapeutics and treatment regimes for the an autoimmune disease or disorder.

In one embodiment, the method described herein promotes the expansion of MDSCs in a subject who is an organ transplant recipient, the method comprising administering a therapeutically effective amount of an agent that activates Tim-3 pathway. The potent immunosuppressive effects of the expanded MDSCs help develop immune tolerance in the host, prevent the immune system from mounting an attack on the transplanted organ, and ensure the long-term viability of the donor organ in the host.

In one embodiment, the method described herein can be administered in conjunction with other immunosuppressive drugs and treatment regime for the organ transplant recipients.

In one embodiment, the method described herein blocks the expansion of MDSCs in a subject who is afflicted with cancer, the method comprising administering a therapeutically effective amount of an agent that inhibits the Tim-3 pathway. In one embodiment, the subject is receiving cancer immunotherapy. Cancer immunotherapy is the use of the immune system to reject cancer. The main premise is stimulating the patient's immune system to attack the malignant tumor cells that are responsible for the disease. This can be either through immunization of the patient, in which case the patient's own immune system is trained to recognize tumor cells as targets to be destroyed, or through the administration of therapeutic antibodies as drugs, in which case the patient's immune system is recruited to destroy tumor cells by the therapeutic antibodies. Since the expansion of MDSCs have been associated with INF-γ, and the immunosuppressive activity of MDSCs undermines cancer immunotherapy, an agent that inhibits the Tim-3 pathway can limit the immunosuppressive effect of MDSCs to give the cancer immunotherapy a chance to work.

In one embodiment, the method described herein can be administered in conjunction with other anti-cancer therapeutics and treatment regimes for the treatment of cancer.

In one embodiment, the method described herein blocks the expansion of MDSCs in a subject whose immune system is compromised, the method comprising administering a therapeutically effective amount of an agent that inhibits the Tim-3 pathway. By inhibiting the expansion of MDSCs, continual suppression of the already weakened immune system is reduced or prevented, thereby allowing the subject to mount a sufficiently effective defense when infectious pathogens are encountered.

It is envisioned that the methods disclosed herein can be applied in conjunction with other treatment methods for autoimmune diseases, transplant organ rejection, cancer, and immunodeficiency.

It is also envisioned that the methods described herein can be used as prophylaxis against pathogen infections disclosed supra, for example, in individuals who are high risk for exposure to pathogens or work in areas with high incidence of pathogen occurrences and infections.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

As used herein, the terms “administering,” refers to the placement of an agent that can modulate the Tim-3 pathways into a subject by a method or route which results in at least partial localization of the agent at a desired site, the Tim-3 expressing Th1 cells. The agent can be administered by any appropriate route which results in an effective treatment in the subject.

As used herein, the term “comprising” or “comprises” is used in reference to methods, and respective component(s) thereof, that are essential to the invention, yet open to the inclusion of unspecified elements, whether essential or not. The use of “comprising” indicates inclusion rather than limitation.

Routes of administration include, but are not limited to, intradermal, intramuscular, intraperitoneal, intravenous, subcutaneous, intranasal, epidural, and oral routes. The agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The precise dose to be employed in the formulation of the agent will also depend on the route of administration, and the seriousness of the disease or disorder, and should be decided according to the judgment of the practitioner and each patient's circumstances. Effective doses may be extrapolated from dose-response curves derived from in vitro or animal model test systems.

Efficacy testing can be performed during the course of treatment using the methods described herein. Measurements of the degree of severity of a number of symptoms associated with a particular ailment are noted prior to the start of a treatment and then at later specific time period after the start of the treatment. For example, when treating an autoimmune disease such as rheumatoid arthritis, the severity of joint pain can be scored from a number of 1-10, with a score of 1 representing mild discomfort and a score of 10 represent constant unbearable pain with or without movement; the range of motion of an affected joint can also are be measured as a degree of angle for which that joint can move. The joint pain and range of motion are noted before and after a treatment. The severity of joint pain and range of motion after the treatment are compared to those before the treatment. A decrease in the pain score and/or an increase in the degree of angle of joint movement indicate that the treatment is effective in reducing inflammation in the affected joint, thereby decreasing pain and improving joint movement.

Alternately, the treatment efficacy can be determined by measuring the population of MDSCs in the subject prior to and after the start of treatment. The population of MDSCs can be determined by FACS analysis using the markers characteristic of MDSCs as disclosed herein from a sample of peripheral blood.

The present invention can be defined in any of the following alphabetized paragraphs:
[A] The use of an agent that modulates a Tim-3 pathway and an acceptable pharmaceutical carrier for modulating a population of myeloid-derived suppressor cells in a subject in need thereof.
[B] The use of an agent that modulates a Tim-3 pathway in the manufacture of a medicament for modulating a population of myeloid-derived suppressor cells in a subject in need thereof.
[C] The use of paragraph [A] or [B], wherein the modulation of the population of myeloid-derived suppressor cells comprises promoting the expansion of myeloid-derived suppressor cells and wherein the agent activates the Tim-3 pathway.
[D] The use of paragraph [C], wherein the agent that activates the Tim-3 pathway is galectin-9.
[E] The use of paragraph [C] or [D], wherein the subject is suffering from an autoimmune disease.
[F] The use of paragraphs [C] or [D], wherein the subject is an organ transplant recipient.
[G] The use of paragraph [A] or [B], wherein the modulation of the population of myeloid-derived suppressor cells comprises preventing the expansion of myeloid-derived suppressor cells and wherein the agent inhibits the Tim-3 pathway.
[H] The use of paragraph [G], wherein the agent that inhibits the Tim-3 pathway comprises an antibody that specifically binds Tim-3.
[I] The use of paragraph [G] or [H], wherein the subject is suffering from cancer.
[J] The use of either of paragraphs [G]-[I], wherein the subject is immunocompromised.
[K] The use of either of paragraphs [G]-[J], wherein the subject is suffering from an infection.
[L] The use of an agent that activates a Tim-3 pathway and expands a population of myeloid-derived suppressor cells in a subject for the treatment of an autoimmune disease in the subject in need thereof.
[M] The use of an agent that activates a Tim-3 pathway and expands a population of myeloid-derived suppressor cells in a subject in the manufacture of a medicament for the treatment of an autoimmune disease in the subject in need thereof.
[N] The use of paragraph [L] or [M], wherein the Tim-3 pathway is activated by galectin-9.
[O] The use of an agent that inhibits a Tim-3 pathway and inhibits an expansion of a population of myeloid-derived suppressor cells in a subject for the treatment of cancer in a subject in need thereof.
[P] The use of an agent that inhibits a Tim-3 pathway and inhibits an expansion of a population of myeloid-derived suppressor cells in a subject in the manufacture of a medicament for the treatment of cancer in a subject in need thereof.
[Q] The use of paragraph [O] or [P], wherein the Tim-3 pathway is inhibited by a Tim-3 antibody.
[R] A method of modulating a population of myeloid-derived suppressor cells in a subject in need thereof, the method comprising administering to the subject a therapeutically effective amount of an agent that modulates the Tim-3 pathway and an acceptable pharmaceutical carrier, whereby a population of myeloid-derived suppressor cells is modulated in said subject.
[S] The method of paragraph [R], wherein the modulation of the population of myeloid-derived suppressor cells comprises promoting the expansion of myeloid-derived suppressor cells and wherein the agent activates the Tim-3 pathway.
[T] The method of paragraph [S], wherein the agent that activates the Tim-3 pathway is galectin-9.
[U] The method of paragraph [S] or [T], wherein the subject is suffering from an autoimmune disease.
[V] The method of paragraphs [S] or [T], wherein the subject is an organ transplant recipient.
[W] The method of paragraph [R], wherein the modulation of the population of myeloid-derived suppressor cells comprises preventing the expansion of myeloid-derived suppression cells and wherein the agent inhibits the Tim-3 pathway.
[X] The method of paragraph [W], wherein the agent that inhibits the Tim-3 pathway comprises an antibody that specifically binds Tim-3.
[Y] The method of paragraph [W] or [X], wherein the subject is suffering from cancer.
[Z] The method of either of paragraphs [W]-[Y], wherein the subject is immunocompromised.
[AA] The method of either of paragraphs [W]-[Z], wherein the subject is suffering from an infection.
[BB] A method of treating an autoimmune disease in a subject in need thereof, the method comprising expanding a population of myeloid-derived suppression cells in a subject comprising activating Tim-3 pathway.
[CC] The method of paragraph [BB], wherein Tim-3 pathway is activated by galectin-9.
[DD] A method of treating cancer in a subject in need thereof, the method comprising inhibiting expansion of a population of myeloid-derived suppression cells in a subject comprising inhibiting Tim-3 pathway.
[EE] The method of paragraph [DD], wherein Tim-3 pathway is inhibited by a Tim-3 antibody.

This invention is further illustrated by the following example which should not be construed as limiting. The contents of all references cited throughout this application, as well as the figures and table are incorporated herein by reference.

### EXAMPLE

### Materials and Methods

Transgenic Mice—For the generation of Tim-3 transgenic mice the full-length cDNA of Tim-3 (Balb/c strain) was cloned into the human CD2 expression cassette ¹⁵ and the construct micro-injected directly into C57BL/6 oocytes. Galectin-9 transgenic ¹⁸ and Tim-3−/−^{12,14} mice were described previously. All animals were housed according to the guidelines established by the Harvard Committee on Animals.

Flow Cytometry—Single cell suspensions from thymus, lymph node or spleen were prepared and stained with the indicated antibodies. Spleens were subjected to digestion with collagenase D (Roche). All flow cytometry data were collected on a BD FACS Calibur (BD Biosciences) and analyzed with FlowJo Software (Tree Star).

In vitro T cell proliferation and measurement of cytokines—Lymphocytes were cultured in triplicate with soluble anti-CD3 in the presence of irradiated antigen presenting cells. For some experiments, CD11b+CD11c− cells were sorted by flow cytometry and used as antigen presenting cells. At 48 h, supernatants were collected for the measurement of cytokines and plates pulsed with [³H]Thymidine and harvested 16 hours later. Cytokines were measured from culture supernatants by either ELISA or cytometric bead array (CBA) (BD Biosciences).

Immunization and assessment of recall responses to TNP-OVA—Mice were immunized subcutaneously with 100 μg of TNP-OVA in CFA. On day 10, draining lymph nodes were harvested and restimulated with TNP-OVA. Proliferation and cytokine production were measured as described above.

Induction and assessment of EAE—Wild-type or Tim-3 transgenic littermates were injected subcutaneously with 100 μg of MOG 35-55 peptide (MEVGWYRSPFSRVVHLYRNGK) (SEQ. ID. No. 1) emulsified in CFA (Difco) supplemented with 4 μg ml-1 Mycobacterium tuberculosis and injected twice intravenously with 100 ng of pertussis toxin (List Biological Laboratories). Clinical assessment of EAE was as follows: 0, no disease; 1, decreased tail tone; 2, hindlimb paresis; 3, complete hindlimb paralysis; 4, forelimb and hindlimb paralysis; 5, moribund state. Numbers of inflammatory foci in meninges and parenchyma were counted and relative numbers of parenchymal neutrophils were assessed in paraffin sections of CNS tissues by a neuropathologist blinded to the genotype and clinical score, as in previous studies 10.

Adoptive transfers—1.5−2×10⁶ sorted CD4+ Tim-3 Tg+ or Tg-cells were injected intravenously into 6 week old Rag1−/− C57BL/6 mice. On day 35-40 post-transfer, spleens were harvested and digested with collagenase D (Roche) prior to cell sorting (BD FACSAria, BD Biosciences) and analysis by flow cytometry.

RNA was isolated using Qiagen RNeasy kit and real time PCR performed for the expression of galectin-9. (5′Gal9: 5′-GTTGTCCGAAACACTCAGAT-3′ (SEQ. ID. No. 2); 3′Gal9: 5′-ATATGATCCACACCGAGAAG-3′ (SEQ. ID. No. 3); probe:5′-DFAM-CAGGAAGAGCGAAGTCTGCT-DTAM-3′ (SEQ. ID. No. 4).

### Results

### Tim-3 Expressed on T Cells Regulates Both Adaptive and Innate Immunity

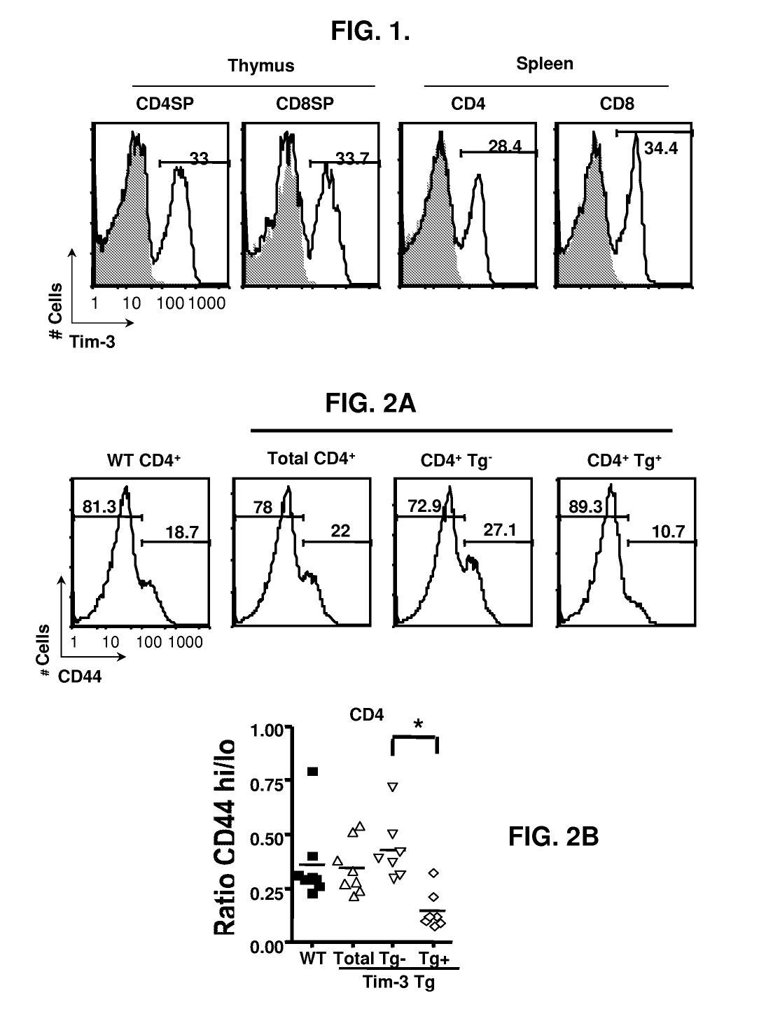

A Tim-3 transgenic mouse (Tim-3 Tg) was generated by expressing the full-length Tim-3 cDNA (BALB/c isoform) under the control of the human CD2 promoter¹⁵ on the C57BL/6 genetic background. With this strategy, Tim-3 transgene positive cells were tracked with an antibody specific for the BALB/c isoform of Tim-3. As expected, the Tim-3 transgene is expressed at the double negative (DN) stage and maintained through the double positive (DP) and single positive (SP) stages of thymocyte development (FIG. 1 and data not shown). Interestingly, the Tim-3 transgene is only expressed in 30-40% of CD4+SP and CD8+SP thymocytes (FIG. 1). Analysis of thymocyte development in Tim-3 Tg mice revealed no major abnormalities (data not shown).

In the peripheral lymphocyte compartment, there were no alterations in the frequency or numbers of T or B cells. As observed in the thymus, only 30-40% of peripheral CD4+T and CD8+T cells express the Tim-3 transgene (FIG. 1). Next, the effector/memory phenotype of peripheral CD4+ and CD8+ T cells were characterized. Comparison of the whole CD4+ and CD8+ T cell compartment of wild type and Tim-3 Tg mice revealed no major differences (FIG. 2 and data not shown). However, when CD4+ T cells from the Tim-3 transgenic mice were segregated into Tim-3 Tg+ and Tim-3 Tg− populations, the Tim-3 Tg+ T cell population contained significantly fewer effector/memory (CD44high) T cells compared to the Tim-3 Tg− population (10% vs. 27%, respectively) (FIG. 2), indicating that Tim-3 expression on T cells controls effector/memory cell generation. The same trend was observed with CD62L expression and in the CD8+ T cells (data not shown). Importantly, the Tim-3 Tg− T cell compartment was not affected in that the ratio of effector/memory (CD44high) to naïve (CD44low) cells was not altered relative to that of wild type littermate controls (FIG. 2B).

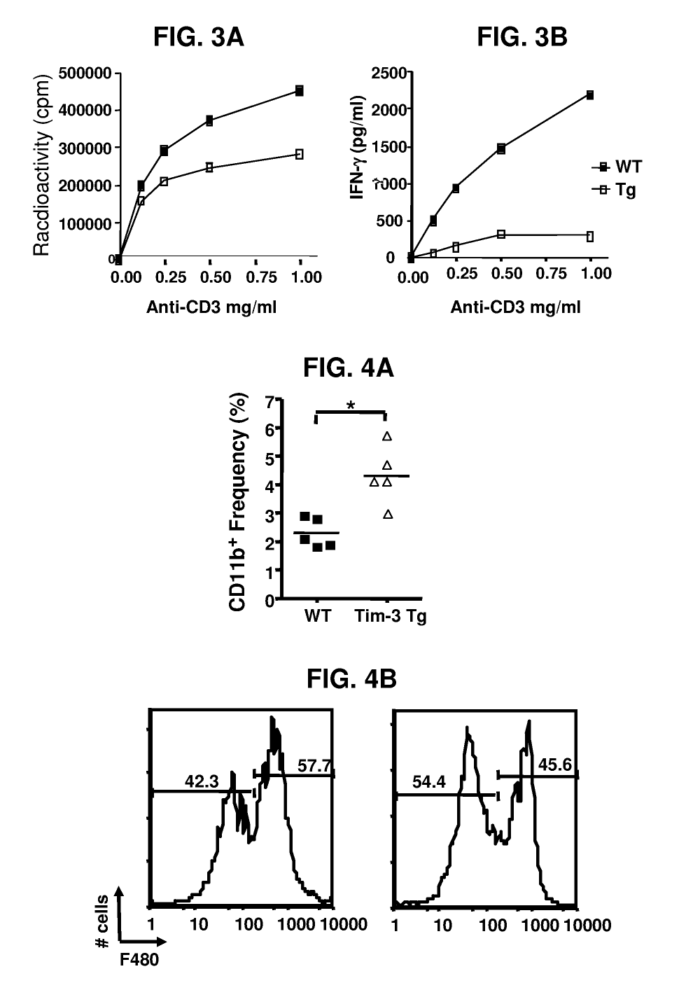

To determine how Tim-3 expression on a limited number of T cells could affect the total T cell response, the response of total splenocytes from the Tim-3 transgenic mice to anti-CD3 activation was tested. Even with the expression of Tim-3 on a limited number of T cells, T cell proliferation and IFN-γ production by the total splenocyte population were clearly reduced compared to wild type mice (FIGS. 3A and 3B). These data indicate that there is a dominant factor(s) in Tim-3 Tg mice that suppresses T cell responses.

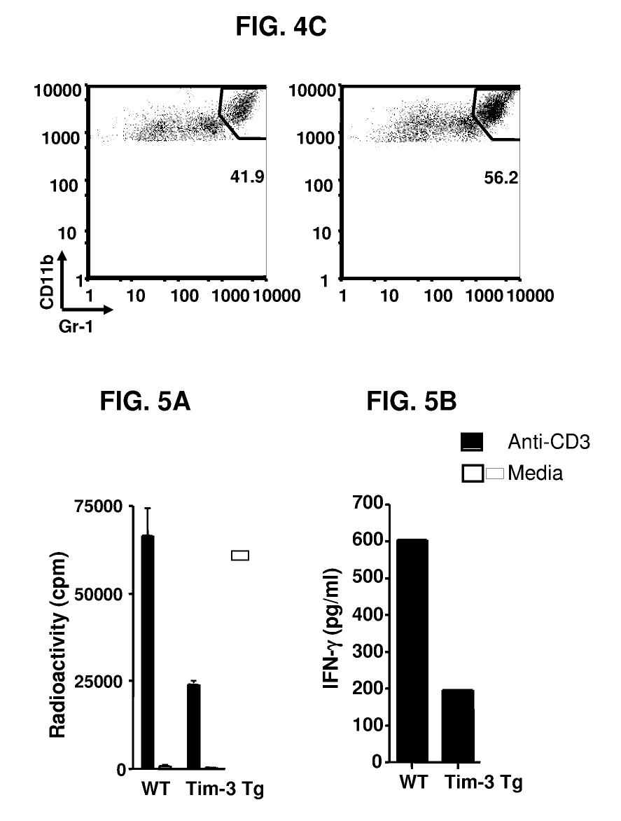

The antigen presenting cell compartment in Tim-3 Tg mice were examined. It was found that the CD11b+ population was significantly increased in Tim-3 Tg mice compared to wild type littermates (FIG. 4A). No significant difference in the frequency or number of dendritic cells (CD11c+) or B cells (CD19+) was observed (data not shown).

The phenotype of the expanded CD11b+ cells in Tim-3 Tg mice was characterized by staining for F480, Ly6G, Ly6C, and MHCII. It was found that the proportion of CD11b+ cells that are F480low and Gr-1/Ly6Ghi is increased in Tim-3 Tg mice relative to wild type littermates (FIGS. 4B and 4C). Since low F480 expression and high Gr-1/Ly-6G expression have previously been associated with MDSCs, this result suggested that there is an increase in the frequency of MDSCs in Tim-3 Tg mice. To test this functionally, the stimulatory capacity of CD11b+ cells from Tim-3 Tg was compared to wild type mice. As shown in FIG. 5A, when compared to their wild type counterparts, purified CD11b+from Tim-3 Tg mice were poor stimulators of both proliferation and IFN-γ secretion from wild type CD4+ T cells (FIG. 5B).

Given that MDSCs are expanded in Tim-3 Tg mice and that their polyclonal T cell responses are suppressed in vitro, the ability to generate any autopathogenic immune responses in vivo was examine. Tim-3 Tg mice were immunized for the development of Experimental autoimmune encephalomyelitis (EAE), a model of central nervous system (CNS) autoimmunity in which myelin-specific effector T cells play a central role in disease induction. It was found that the incidence of clinical EAE was significantly reduced in Tim-3 Tg mice compared to wild type littermates (36% and 93%, respectively). This was consistent with the idea that transgenic expression of Tim-3 on T cells affected the capacity of the animals to mount a pathogenic T cell response. Histological examination revealed that the Tim-3 Tg mice with no clinical score showed very few lesions or inflammatory foci in the CNS (Table 1).

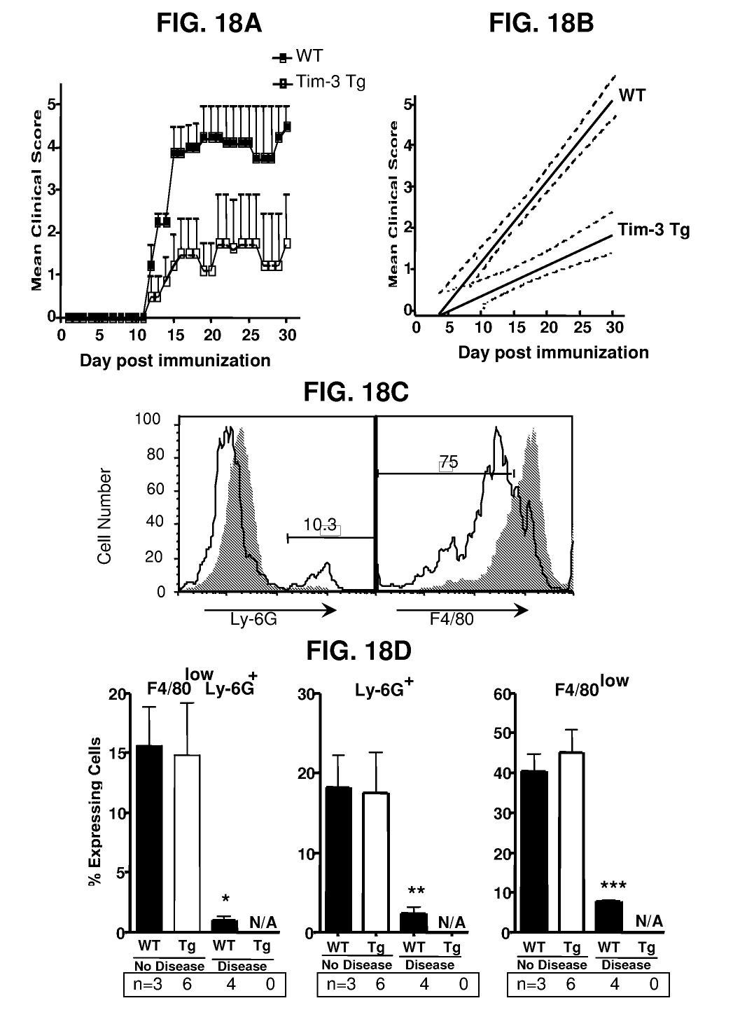

Tim-3 Tg (n=4) and wild type littermates (n=4) were immunized with 100 μg of MOG 35-55 in CFA and administered 100 ng pertussis toxin intravenously on Days 0 and 2. Immunized mice were monitored for the development of EAE. It was essentially shown that the presence of MDSC, that is CD11b+F480 low Ly-6G+ cells correlates with the absence of clinical disease in the EAE model of autoimmunity (FIG. 18). Accordingly, collectively these data support a model where transgenic Tim-3 expression inhibits the generation of pathogenic T cells directly and also indirectly by promoting the expansion of MDSCs that in turn suppress the generation of autopathogenic T cell responses. Given that the majority of T cells in Tim-3 Tg mice do not express the Tim-3 transgene and thus are not subject to galectin-9-mediated regulation, these data further indicate that the role of Tim-3 in promoting the expansion of MDSCs is a dominant factor in the suppression of autopathogenic T cell responses.

### T-Cell Expression of Tim-3 Promotes the Expansion of MDSC

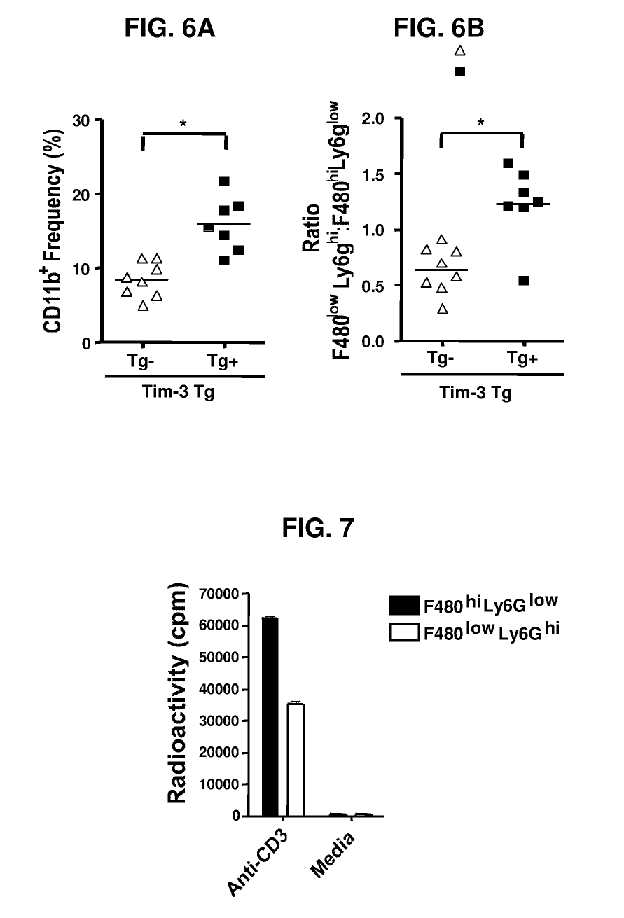

To determine whether Tim-3 expression on T cells triggers the expansion of MDSCs in vivo, CD4+ Tim-3 Tg+ and CD4+ Tim-3 Tg− were isolated from Tim-3 Tg mice and were then transferred into Rag1−/− recipients which lack T and B cells but have an intact myeloid compartment. After 4-6 weeks, Rag1−/− mice reconstituted with CD4+ Tim-3 Tg+ T cells contained significantly higher percentages of CD11b+ cells that were F480^{low}Ly6G^{hi} than mice reconstituted with CD4+ Tim-3 Tg− T cells, indicating that a constitutive expression of Tim-3 on T cells could induce the expansion of MDSCs (FIG. 6). Functional analysis of the CD11b+F480^{low}Ly6G^{hi} cells from the recipients revealed that these MDSCs are indeed poor stimulators of T cell proliferation (FIG. 7). Collectively, these data show that Tim-3, when expressed on T cells, triggers the expansion of a suppressive population of myeloid-derived cells characterized as CD11b+F4/80^{low}Ly6G^{hi} cells. The higher frequency of MDSCs likely underlies the reduction in the generation of effector pathogenic T cells and the overall reduced susceptibility to EAE observed in Tim-3 Tg mice.

### T Cell Responses in Galectin-9 Transgenic Mice

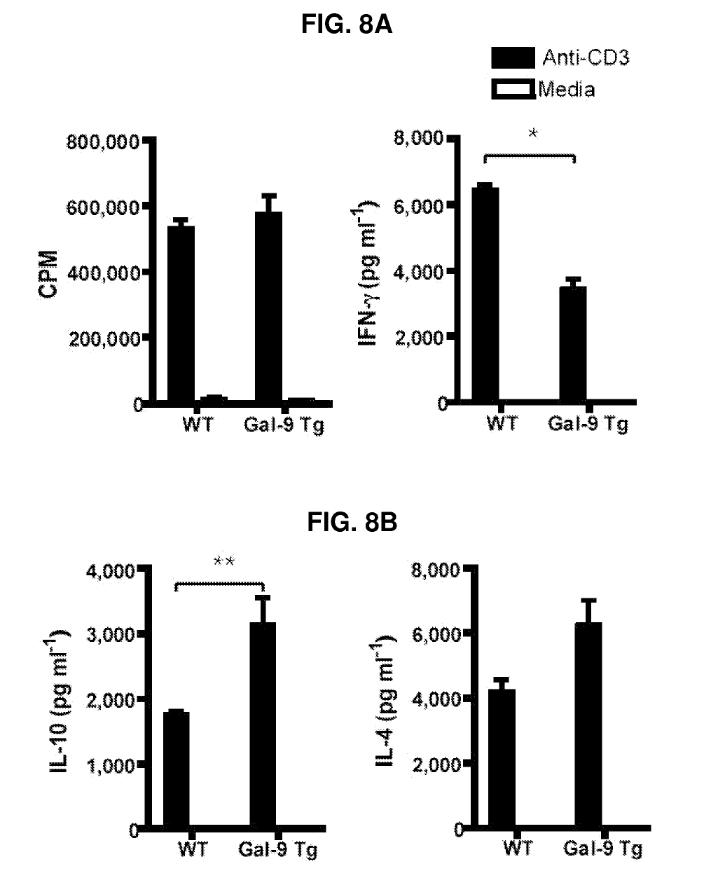

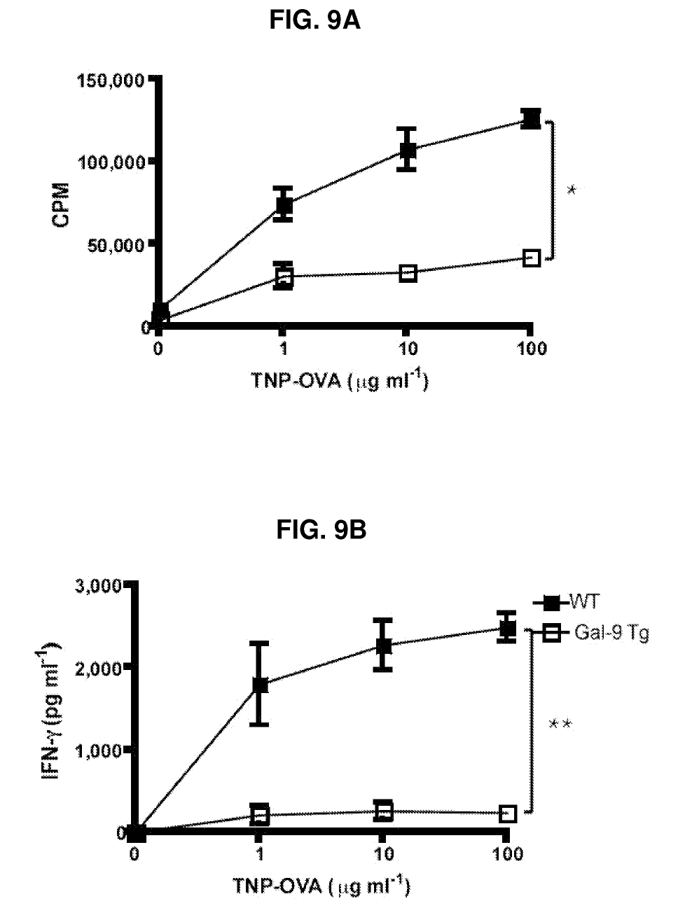

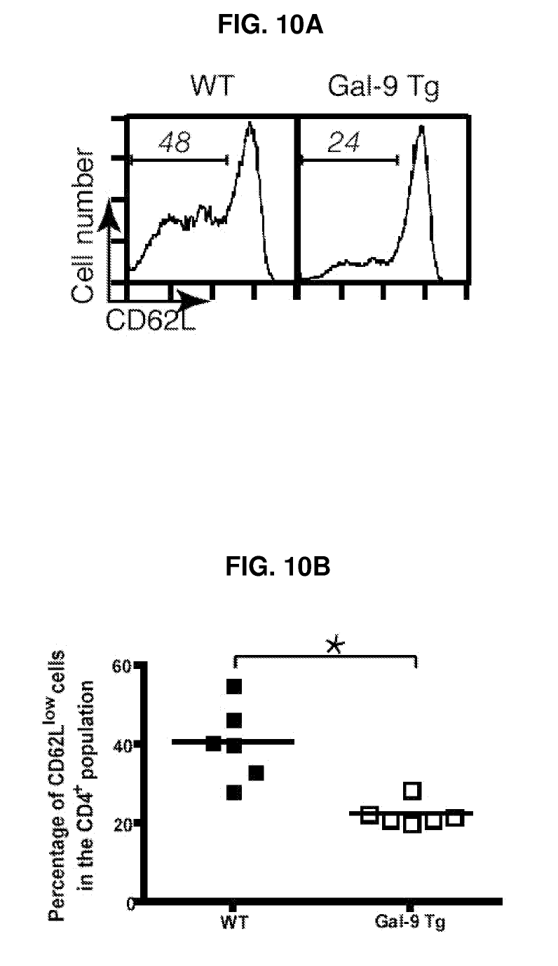

Given that galectin-9 is a ligand for Tim-3, galectin-9 overexpression was examined to determine if it were similarly involved in the inhibition of T cell responses and the generation of MDSCs. Therefore, the ability of T cells from galectin-9 transgenic (Gal-9 Tg) mice to proliferate and produce cytokines were examined. When activated with anti-CD3 in vitro, no significant differences in either T cell proliferation or cell death between Gal-9 Tg mice and wild type littermate controls were observed (FIG. 8A and data not shown). However, there was a significant decrease in IFN-γ production (FIG. 8A) and a concomitant increase in IL-10 and IL-4 (FIG. 8B) production in spleen cells from Gal-9 Tg mice compared to wild type littermate controls. The in vivo relevance of these findings by immunizing Gal-9 Tg mice and wild type littermates with TNP-OVA was examined next. It was found that cells from immunized Gal-9 Tg mice exhibited a dramatic decrease in the ability to proliferate (FIG. 9A) and produce IFN-γ (FIG. 9B) upon in vitro reactivation with TNP-OVA. IL-4 and IL-10 were not produced (data not shown). In the Gal-9 Tg immunized mice, there was a significant decrease in the CD4+CD62Llow effector/memory T cell population (FIG. 10). Thus, similarly to Tim-3 Tg mice, there is a marked decrease in the ability of Gal-9 Tg mice to prime Th1 immune responses and generate effector/memory cells.

### Expression of Tim-3 Ligand, Galectin-9, Promotes the Expansion of MDSCs

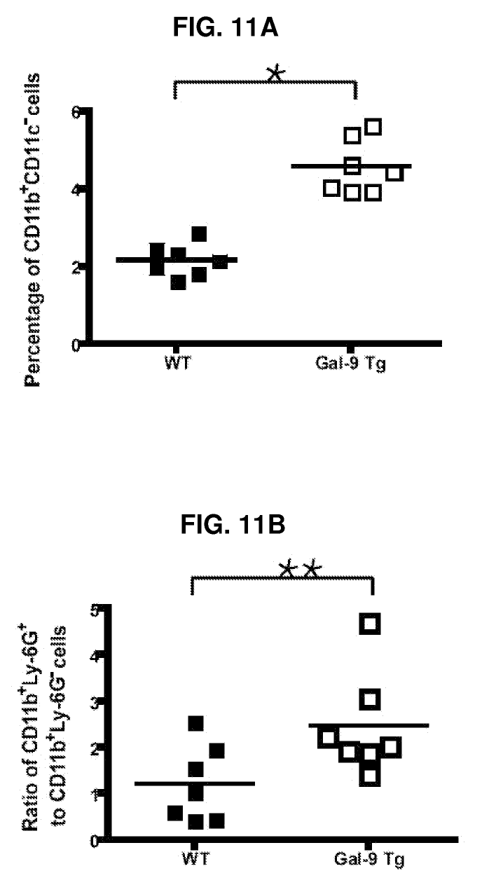

Given the observation that MDSCs are expanded in Tim-3 Tg mice, the population of MDSCs was examined in Gal-9 Tg mic and was found to be similarly expanded. There was a significant increase in the CD11b+ myeloid population in Gal-9 Tg mice (FIG. 11A) but no significant differences in the numbers/frequency of dendritic cells (CD11c+) or B cells (CD19+) (data not shown). Further characterization of the CD11b+ cells from Gal-9 Tg mice revealed an increase in the proportion of MDSCs (CD11b+Gr-1/Ly6G+F4/80low) in Gal-9 Tg mice (FIG. 11B). Altogether these data demonstrate a significant expansion of MDSCs in mice overexpressing galectin-9 and indicate an important role for galectin-9 in the development of these cells.

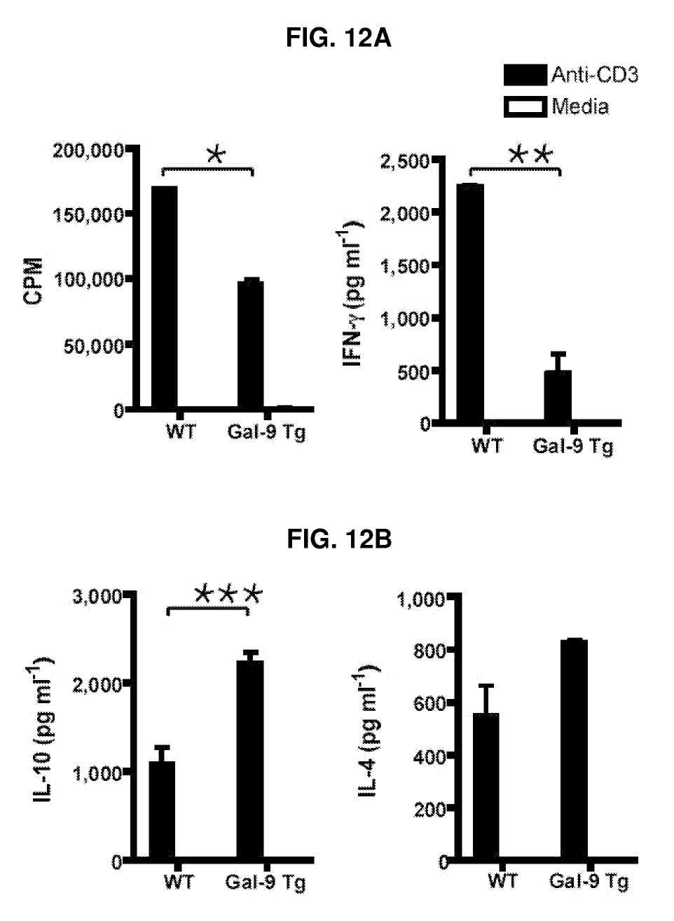

To determine if the expansion of MDSCs may be responsible for the defect in priming Th1 immune responses in Gal-9 Tg mice, the T cell responses elicited in vitro by the CD11b+ cells obtained from galectin-9 transgenic mice using anti-CD3 as the stimulus were determined. There was a marked decrease in proliferative responses when using CD11b+ cells from Gal-9 Tg mice as APC (FIG. 12A). This was accompanied by an almost total absence of IFN-γ production (FIG. 12A) with a concomitant increase in IL-4 and IL-10 production (FIG. 12B). Collectively, these data indicate that that the expansion of MDSCs could be responsible for the defect in productive Th1 immune responses observed in galectin-9 transgenic mice in response to immunization. Since galectin-9 transgenic mice are on the BALB/c background, it was not possible to examine the role of the galectin-9 transgene on EAE.

### Galectin-9/Tim-3 Interaction in the Development of MDSCs

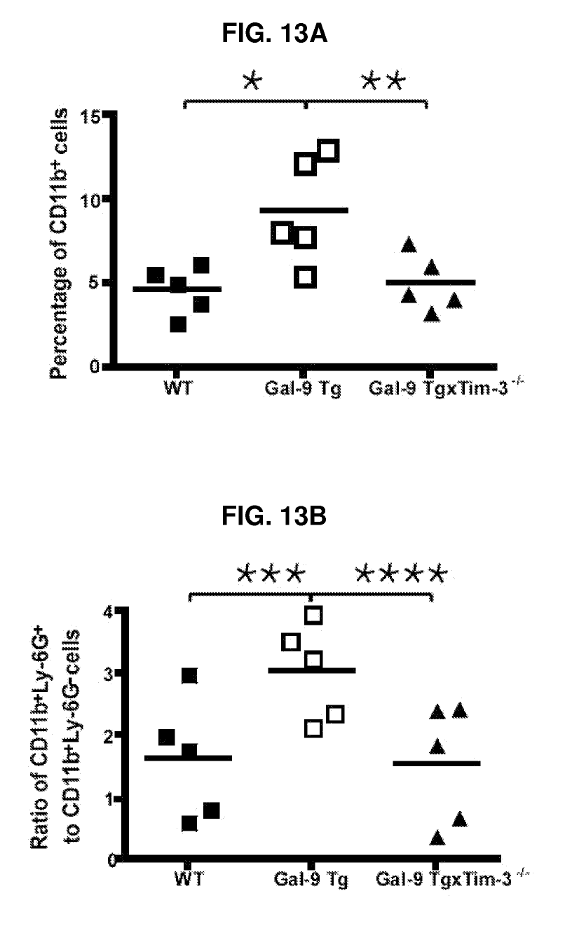

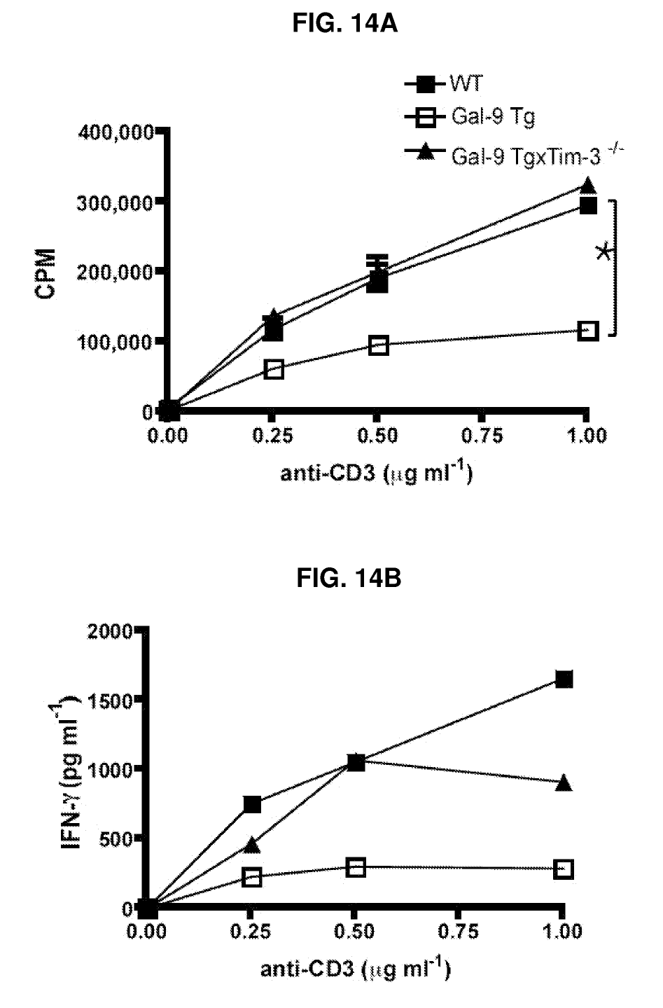

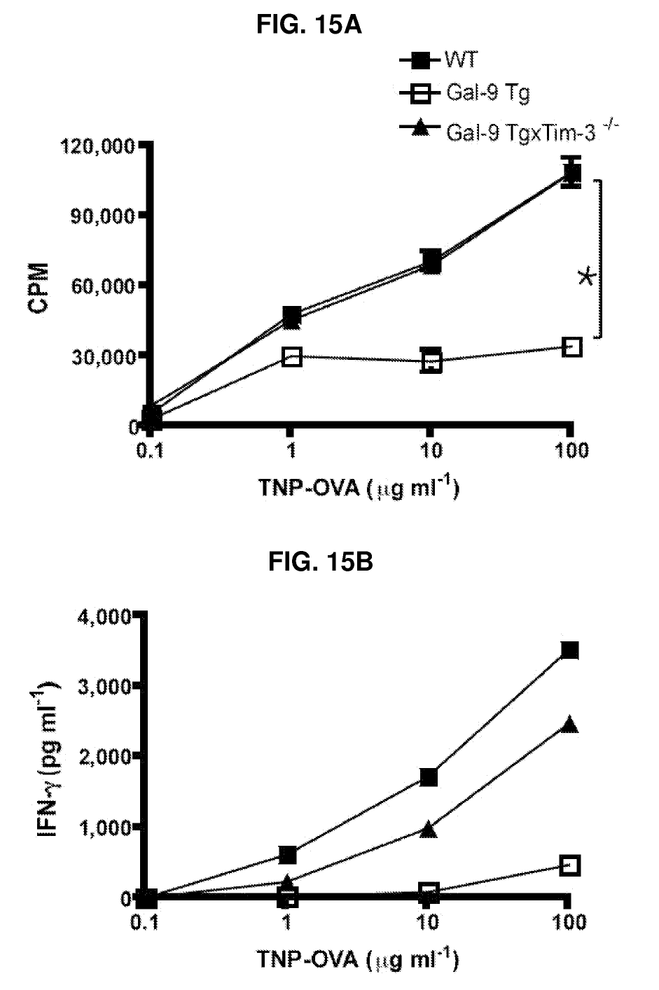

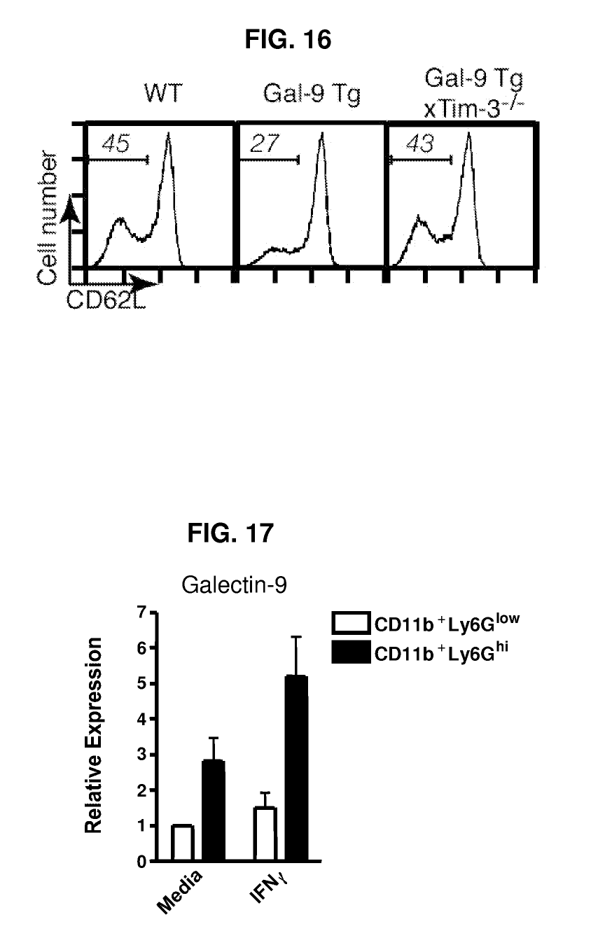

Since transgenic expression of both Tim-3 and its ligand, galectin-9, independently resulted in the expansion of MDSCs, this raised the issue of whether a direct interaction of Tim-3 with galectin-9 is responsible for the expansion of MDSCs that was observed in the transgenic mice. To test this, Gal-9 Tg mice were crossed with Tim-3^{-/-} mice. Comparison of Gal-9 Tg+ (Gal-9 Tg), Gal-9 Tg-(WT), and Gal-9 Tg+×Tim-3^{-/-} mice revealed that the increased frequency of CD11b+ cells observed in the Gal-9 transgenic mice was restored to wild type levels in Gal-9 Tg×Tim-3^{-/-} mice (FIG. 13A). Further characterization of the CD11b+ cells obtained from the different mouse strains demonstrated that the proportion of CD11b+ cells expressing the MDSC phenotype is also reversed to wild type levels in Gal-9 Tg×Tim-3^{-/-} mice, thereby confirming that the expansion of MDSC in Gal-9 Tg mice is dependent on the interaction with Tim-3 (FIG. 13). Moreover, the CD11b+ cells from Gal-9 Tg×Tim-3^{-/-} mice were equally able to stimulate proliferation and IFN-γ production from wild type CD4+ T cells as CD11b+ cells taken from wild type mice (FIG. 14). Similarly, the priming of Th1 immune responses was restored to wild type levels in Gal-9 Tg×Tim-3−/− mice immunized with TNP-OVA (FIG. 15). Lastly, the defect in the generation of CD62L low effector/memory cells in Gal-9 Tg mice following immunization was also corrected in immunized Gal-9 Tg×Tim-3−/− mice such that the Gal-9 Tg×Tim-3−/− mice had close to wild type levels of effector/memory T cells (FIG. 16).

Data presented here demonstrate that the generation of productive Th1 immune responses is suppressed in both Tim-3 Tg and Gal-9 Tg mice. Given that the majority (60-70%) of T cells in the Tim-3 Tg mice do not express Tim-3, this global defects in T cell responsiveness, IFN-γ production and are relatively resistant to the development of EAE is surprising. This is consistent with a model whereby Tim-3 inhibits Th1 responses at two different levels: “cell intrinsic” in which the expression of Tim-3 on terminally differentiated Th1 cells induces cell death by crosslinking with galectin-9 and “cell extrinsic” where the Tim-3/galectin-9 pathway promotes the expansion of MDSCs which in turn suppresses T cell responses. Indeed, it is believed that the Tim-3/galectin-9 pathway has evolved to control inflammatory T cell responses by multiple means: the specific induction of cell death in terminally differentiated Th1 cells, previously proposed ¹² and the hitherto unknown pathway of expansion of myeloid-derived suppressor cells from pre-existing precursors. This raises the issue as to why the Tim-3:galectin-9 pathway expands MDSCs? Preliminary data demonstrate that CD11b+Ly-6G+ cells with MDSC phenotype exhibit the highest galectin-9 expression and this may be one of the mechanisms by which Tim-3 expression on terminally differentiated Th1 cells (or transgenic T cells) expands MDSCs (FIG. 17).

Taken together, these new data elucidate a novel function of the galectin-9/Tim-3 pathway in the expansion of CD11b+Gr-1/Ly6G+F4/80^{low}MHCII^{low} myeloid-derived suppressor cells and shed some light on the previously unexplained association of IFN-γ with MDSC activation/expansion. Since terminally differentiated IFN-γ-producing Th1 cells express Tim-3 on their cell surface, an interaction between Tim-3 expressed on Th1 cells and galectin-9 expressed on MDSCs likely signals their expansion. The galectin-9/Tim-3 pathway represents the first cell surface receptor/ligand pair identified in playing a role in MDSC expansion. Since MDSCs have been demonstrated to be pivotal regulators of CD4+ and CD8+ T cell responses in cancer ^{7,8}, bacterial ^{1,2}, parasitic ^{3,4}, and viral ⁵ diseases and of beneficial value in regulating autoimmune diseases ¹⁶′¹⁷ the galectin-9/Tim-3 pathway represents an important therapeutic target for the treatment of multiple diseases.

The references cited herein and throughout the specification are incorporated herein by reference.

### REFERENCES

1. Cauley, L. S., Miller, E. E., Yen, M. & Swain, S. L. Superantigen-induced CD4 T cell tolerance by myeloid cells and IFN-gamma. J. Immunol. 165, 6056-66. (2000).
2. Delano, M. J. et al. MyD88-dependent expansion of an immature Gr-1+ CD11b+ population induces T cell suppression and Th2 polarization in sepsis. J. Exp. Med. 204, 1463-74. (2007).
3. Atochina, O., Daly-Engel, T., Piskorska, D., McGuire, E. & Ham, D. A shistosome-expressed immunomodulatory glycoconjugate expands peritoneal Gr1+ macrophages that suppress naive CD4+ T cell proliferation via an IFN-g and nitric oxide dependent mechanism. J. Immunol. 167, 4293-302. (2001).
4. Goni, O., Alcaide, P. & Fresno, M. Immunosuppression during acute Trypanosoma cruzi infection: involvement of Ly6G (Gr1+) CD11b+ immature myeloid suppressor cells. Intl. Immunol. 14, 1125-1134 (2002).
5. Bronte, V. et al. Apoptotic death of CD8+ T lymphocytes after immunization: induction of a suppressive population of Mac-1+/Gr-1+ cells. J. Immunol. 161, 5313-20. (1998).
6. Makarenkova, V. P., Bansal, V., Matta, B. M., Perez, L. A. & Ochoa, J. B. CD11b+/Gr-1+ myeloid suppressor cells cause T cell dysfunction after traumatic stress. J. Immunol. 176, 2085-94. (2006).
7. Kusmartsev, S. & Gabrilovich, D. I. Role of immature myeloid cells in mechanisms of immune evasion in cancer. Cancer Immunol. Immunother. 55, 237-245 (2006).
8. Kusmartsev, S. A., Li, Y. & Chen, S. H. Gr-1+ myeloid cells derived from tumor-bearing mice inhibit primary T cell activation induced through CD3/CD28 costimulation. J. Immunol. 165, 779-85. (2000).
9. Gallina, G. et al. Tumors induce a subset of inflammatory monocytes with immunosuppressive activity on CD8+ T cells. J. Clin. Invest. 116, 2777-90. (2006).
10. Monney, L. et al. Th1-specific cell surface protein Tim-3 regulates macrophage activation and severity of an autoimmune disease. Nature. 415, 536-41. (2002).
11. Anderson, A. C. et al. Promotion of tissue inflammation by the immune receptor Tim-3 expressed on innate immune cells. Science. 318, 1141-43. (2007).
12. Zhu, C. et al. The Tim-3 ligand galectin-9 negatively regulates T helper type 1 immunity. Nat. Immunol. 6, 1245-52. (2005).
13. Sabatos, C. A. et al. Interaction of Tim-3 and Tim-3 ligand regulates T helper type 1 responses and induction of peripheral tolerance. Nat. Immunol. 4, 1102-10. (2003).
14. Sanchez-Fueyo, A. et al. TIM-3 inhibits T helper type 1-mediated auto- and alloimmune responses and promotes immunological tolerance. Nat. Immunol. 4, 1093-101. (2003).
15. Lang, G. et al. The structure of the human CD2 gene and its expression in transgenic mice. Embo. J. 7, 1675-82. (1988).
16. Zhu, B. et al. CD11b+Ly-6Chi suppressive monocytes in experimental autoimmune encephalomyelitis. J. Immunol. 179, 5228-37. (2007).
17. Marhaba, R. et al. The importance of myeloid-derived suppressor cells in the regulation of autoimmune effector cells by a chronic contact eczema. J. Immunol. 179, 5071-81. (2007).
18. Tsuboi, Y. et al. Galectin-9 protects mice from the Shwartzmann reaction by attracting prostaglandin E2-producing polymorphonuclear leukocytes. Clin. Immunol. 124, 221-33. (2007).

TABLE 1
EAE in wildtype and Tim-3 Tg mice.

| | | | | Mean | | | |
|---|---|---|---|---|---|---|---|
| | | | Mean day of | maximum | Mice with clinical disease | | |
| | Inci- | Mortal- | onset ± | score ± | Inflammatory lesions (±s.e.m.) | | |
| Group | dence | ity | sem | sem | Meninges | Parenchyma | Total (n) |
| WT | 13/14 | 3/14 | 14.3 ± 0.6 | 3.1 ± 0.35 | 23.4 ± 5.7 | 22.6 ± 6.2 | 46 ± 11.4 (10) |
| | (93%) | (21%) | | | | | |
| Tim-3 Tg | 5/14 | 1/14 | 13.8 ± 0.49 | 3.1 ± 0.51 | 60.8 ± 13.1 | 59.8 ± 11.7 | 128 ± 22 (4) |
| | (36%)* | (7%) | | | | | |

| | Mice without clinical disease | | |
|---|---|---|---|
| | Inflammatory lesions (±s.e.m.) | | |
| Group | Meninges | Parenchyma | Total (n) |
| WT | 0 | 0 | 0 (1) |
| Tim-3 Tg | 4.3 ± 2.8 | 2.8 ± 1.6 | 7.1 ± 4.4 (9) |

Mice were immunized with MOG 35-55 in complete Freund's adjuvant and monitored for the development of EAE.
*p = 0.0422, paired t-test.

1-17. (canceled)

18. A method of modulating a population of myeloid-derived suppressor cells in a subject in need thereof, the method comprising administering to the subject a therapeutically effective amount of an agent that modulates the Tim-3 pathway and an acceptable pharmaceutical carrier, whereby a population of myeloid-derived suppressor cells is modulated in said subject.

19. The method of claim 18, wherein the modulation of the population of myeloid-derived suppressor cells comprises promoting the expansion of myeloid suppressor cells and wherein the agent activates the Tim-3 pathway.

20. The method of claim 19, wherein the agent that activates the Tim-3 pathway is galectin-9.

21. The method of claim 19, wherein the subject is suffering from an autoimmune disease.

22. The method of claim 19, wherein the subject is an organ transplant recipient.

23. The method of claim 18, wherein the modulation of the population of myeloid-derived suppressor cells comprises preventing the expansion of myeloid-derived suppression cells and wherein the agent inhibits the Tim-3 pathway.

24. The method of claim 23, wherein the agent that inhibits the Tim-3 pathway comprises an antibody that specifically binds Tim-3.

25. The method of claim 23, wherein the subject is suffering from cancer.

26. The method of claim 23, wherein the subject is immunocompromised.

27. The method of claim 23, wherein the subject is suffering from an infection.

28. A method of treating an autoimmune disease in a subject in need thereof, the method comprising expanding a population of myeloid-derived suppression cells in a subject comprising activating Tim-3 pathway.

29. The method of claim 28, wherein Tim-3 pathway is activated by galectin-9.

30. A method of treating cancer in a subject in need thereof, the method comprising inhibiting expansion of a population of myeloid-derived suppression cells in a subject comprising inhibiting Tim-3 pathway.

31. The method of claim 30, wherein Tim-3 pathway is inhibited by a Tim-3 antibody.


SEQUENCE LISTING (4 sequences)

SEQ ID NO: 1 - protein, 21 aa, Unknown
  Description of Unknown MOG 35-55 peptide
MEVGWYRSPFSRVVHLYRNGK

SEQ ID NO: 2 - DNA, 20 bp, Artificial Sequence
  Description of Artificial Sequence Synthetic primer
gttgtccgaaacactcagat

SEQ ID NO: 3 - DNA, 20 bp, Artificial Sequence
  Description of Artificial Sequence Synthetic primer
atatgatccacaccgagaag

SEQ ID NO: 4 - DNA, 20 bp, Artificial Sequence
  Description of Artificial Sequence Synthetic probe
caggaagagcgaagtctgct